(19) United States
(12) Patent Application Publication
(10) Pub. No.: US 2021/0238679 A1
(43) Pub. Date: Aug. 5, 2021

(54) Use of Combinatorial Non-Coding DNA Snippets as Taggants in Consumer Products and Supply Chains

(71) Applicant: SafeTraces, Inc., Pleasanton, CA (US)

(72) Inventors: Quin Chou, Pleasanton, CA (US); Peter Mattei, Lemon Grove, CA (US); Ulrike W. Hodges, Berkeley, CA (US)

(21) Appl. No.: 17/164,632

(22) Filed: Feb. 1, 2021

Related U.S. Application Data
(60) Provisional application No. 62/968,781, filed on Jan. 31, 2020.

(51) Int. Cl. C12Q 1/6876; G06F 16/27

(52) U.S. Cl. CPC C12Q 1/6876; G06F 16/27

(57) ABSTRACT

A method for tagging items comprising applying a plurality of non-coding DNA tags, wherein the selection of the particular taggants corresponds with a binary or nonbinary code sequence containing information about the tagged items.

### CROSS-REFERENCES TO PRIORITY AND RELATED APPLICATIONS

This application claims the benefit of, and priority from, U.S. Provisional patent Application No. 62/968,781 filed Jan. 31, 2020, entitled “Use of Combinatorial Non-coding DNA Snippets as Taggants in Consumer Products and Supply Chains.”

The entire disclosures of the application recited above is hereby incorporated by reference, as if set forth in full in this document, for all purposes.

### FIELD

The present disclosure generally relates to facilitating the tracking of a substance as it moves through a supply and/or distribution chain. The disclosure relates more particularly to apparatus and techniques for using DNA sequences for tracking a material and identifying a material, for example to confirm its source, which can be applicable to a range of industries involving consumer products, including, for example, the pharmaceutical industry, the baby formula industry, the cosmetics industry, the vitamins and supplements industry, the nutraceuticals industry, the personal care industry, and others. These can include tracking source and/or sanitation practices and/or production, supply, and distribution chain monitoring. The disclosure also relates to apparatus and techniques for using non-coding DNA sequences with formulations for source tracking, sanitation monitoring, and production, supply, and distribution chain monitoring.

### BACKGROUND

Products which are destined to reach an end user often have many stops along the supply chain. Similarly, the starting materials used to create the products often have many stops along the production chain, beginning from the raw material source and traveling to the point at which the product is packaged. For example, a pharmaceutical product travels through many channels from the location where an ingredient or compound originates, to where a tablet/pill containing the ingredient or compound is formulated and pressed, to the destination where it is ultimately administered/consumed.

There is a need for technologies which enable tracking commercial products along the production, supply, and distribution chains.

### SUMMARY

Non-coding DNA sequences form tags (or tag sequences) and a value can be encoded with tag sequences, such as where the presence of one tag sequence indicates a “1” in one binary position, the absence of that one tag sequence indicates a “0” in that binary position, and the set of presences and absences of tag sequences associated with various binary positions forms a binary word that provides information about the item in, or on, where the tag sequences are found. The non-coding DNA sequences might be taken from seaweed DNA or other DNA that contains sequences uncommon in foods or marked objects. The sequences are small enough that they would not be coding sequences. A taggant that is applied to a consumer product might comprise a plurality of the non-coding DNA sequences. The consumer product could be, for example, a pharmaceutical product, e.g. a tablet or pill, baby formula, a cosmetics product, a vitamin or supplement, a nutraceutical product, a personal care product, such as a cream or lotion, and the like. The taggant could be provided as a complex with a coating for a tablet or pill, or the taggant could be added directly to the product matrix or formulation. In some embodiments, the DNA can be held and/or maintained and/or encapsulated and/or retained and/or present in a liposomal and/or micellar structure. The DNA-taggant in a liposomal and/or micellar structure can be present in the coating solution or in the product matrix or formulation. This complex can be added directly or applied or delivered to a product in powder form, mixed with water, alcohol, wax, or some other base, and/or encapsulated.

Applying the taggant to the consumer product or object labels the consumer product or object, in a safe and acceptable way, where the label can be in the form of a binary word having some word length with each bit of the binary word having a bit position in the binary word. According to perhaps a predetermined convention, particular non-coding DNA sequences correspond to particular bit positions, and the presence or absence of one of those non-coding DNA sequences indicates a bit value in a particular bit position of the label applied to the consumer product or object. The presence of the tags can later be detected using DNA PCR or other techniques. With those techniques, very little of the taggant is needed for the tag label to be recognizable.

In other variations, the label comprises other than binary bits at each bit position. In some variations, the non-coding DNA sequences include a static portion that is a sequence of nucleotides that is common to all of the non-coding DNA sequences and a variable portion that distinguishes each non-coding DNA sequence from the other non-coding DNA sequences.

In some uses, the taggant in effect labels a consumer product or item at the source of production. In the case of a pharmaceutical product, e.g. a tablet or pill, baby formula, a cosmetics product, a vitamin or supplement, a nutraceutical product, a personal care product, such as a cream or lotion, and the like product, the taggant can be detected at the point of administration/consumption, even if all packaging is removed. In some uses, the taggant serves as a signal for sanitation practices, such as where the taggant is applied to a surface that is to be sanitized and presence of any, or more than a threshold amount of, taggant is an indication of inadequate sanitation.

The label applied by the taggant can represent an ingredient or manufacturing facility identity, a product identifier, a time/date of production, or other data determinable when the taggant is applied.

In one method, items are tagged by having applied thereon a plurality of non-coding DNA tags, wherein a selection of particular tags corresponds with a binary or nonbinary code sequence containing information about tagged items and wherein a non-coding DNA tag comprises a DNA sequence that would not otherwise be present in a tagged item. The DNA tags are non-coding in that they are not coding sequences of DNA that might be part of a cellular operation of coding for protein production and other uses of coding DNA. The items tagged might be consumer products. The items tagged might be pharmaceutical products, e.g. tablets or pills, baby formula, cosmetics products, vitamins or supplements, nutraceutical products, personal care products, such as cream or lotions, and the like. The information might include information as to a source of those products, such as pharmaceutical products, e.g. tablets or pills, baby formula, cosmetics products, vitamins or supplements, nutraceutical products, personal care products, such as cream or lotions, and the like. In another variation, the items tagged are surfaces requiring sanitary handling and the information includes information as to whether the surfaces were sanitized sufficiently.

The DNA tags can be selected from among a set of DNA tags that represents and/or corresponds to a label that is a binary word with bits in bit positions corresponding to whether a particular DNA tag was selected, and wherein the DNA tags of the selection are combined with a carrier to form a taggant that is applied to surfaces requiring sanitary handling or items to be tagged. For example, within a particular vendor or operator's system, if information is representable by an N-bit value, an item can be tagged by DNA tags selected from a set of N DNA tags by applying those DNA tags of the set that correspond to one bit value of the N-bit value (e.g., where a “1” is present in a bit position i in the N-bit value, the i-th DNA tag of the set of DNA tags is included in the material applied to the tagged item and where a “0” is present in a bit position i in the N-bit value, the i-th DNA tag of the set of DNA tags is not included in the material applied to the tagged item, or other variation). In some embodiments, instead of the i-th bit being represented by the presence or absence of the i-th DNA tag of the set of DNA tags, there are 2N DNA tags in the set of DNA tags, with one DNA tag (the i-th “0” tag) being applied to the tagged item if “0” is in bit position i in the N-bit value and another DNA tag (the i-th “1” tag) being applied to the tagged item if “1” is in bit position i in the N-bit value. Other than binary encoding is possible.

The DNA tags can be applied as a complex with the coating of a pill or tablet, such as pharmaceutical products, vitamins or supplements, nutraceutical products, and the like. Alternatively, the DNA tags can be added directly to the product matrix or formulation, as in the case of pharmaceutical formulations, baby formula, cosmetics products, formulations for vitamins or supplements, formulations for nutraceutical products, personal care products, such as cream or lotions, and the like. In some embodiments, the DNA can be held and/or maintained and/or encapsulated and/or retained and/or present in a liposomal and/or micellar structure. The DNA-taggant in a liposomal and/or micellar structure can be present in the coating solution or in the product matrix or formulation. This complex can be added directly or applied or delivered to a product in powder form, mixed with water, alcohol, wax, or some other base, and/or encapsulated

For example, a set of taggants to apply to a product using miniDART can be generated, and a consumer product can then be “tagged” using the selected set of taggants (where the set might represent a 32-bit “DNA barcode”). The taggants can be encapsulated by mixing taggants with a consumer product component to form a liposomal or micellar structure. The DNA taggant-containing liposomal or micellar structure can then be used as a component in a pill/tablet coating; appropriate coating components, combinations of components, ratios of components, and formulations would be appreciated by those skilled in the art. The DNA taggant-containing liposomal or micellar structure can alternatively then be added directly to the product matrix or formulation.

For example, in the case of use as a component in a pill/tablet, taggants can be encapsulated by mixing taggants with carrier gel-phase HPMC and polysorbate 80, a surfactant. An exemplary coating mixture in accordance with some embodiments includes EUDRAGIT 30D, HPMC, Polysorbate 80 and talc to generate the DNA tagged coating solution for spraying onto the pills. Taggants particles as described above enhance the binding of taggants to consumer products and can easily be removed from equipment surfaces by a standard sanitation procedure. The encapsulated taggants can optionally include fluorescing compositions for quickly monitoring the presence or non-presence of taggants. The fluorescing compositions are stable, do not leave a mark, are non-visible under normal light, and fluoresce only under UV light. The fluorescing dye can be optical brightener. A stable water-soluble solution of self-assembled carriers can be directed through the appropriate dosing equipment with minimal or no accumulation within production devices. Delivery can optionally involve a further carrier, such as air, water, alcohol or other volatile substance, a wax, a powdering agent, and/or microbeads.

In some variations, information relating to the tagging and/or labeling process are recorded, in a public blockchain and might include one or more of a time of production, a name of a company, production details, a type of ingredient/component, a supervisor name, a batch size, an expected customer, a serial number of a taggant dispenser, a label assigned to a batch, a code alphabet, error correction used, a taggant suspension type, and/or sequences of DNA nucleotides used for the plurality of non-coding DNA tags.

A method is described for tagging items comprising applying a plurality of non-coding DNA tags, wherein a selection of particular tags corresponds with a binary or nonbinary code sequence containing information about the items.

An apparatus is described for tagging items comprising applying a plurality of non-coding DNA tags, wherein a selection of particular tags corresponds with a binary or nonbinary code sequence containing information about the items.

A reading apparatus might be used for reading tags from tagged items tagged with a plurality of non-coding DNA tags, wherein a selection of particular tags corresponds with a binary or nonbinary code sequence containing information about the tagged items.

The following detailed description together with the accompanying drawings will provide a better understanding of the nature and advantages of the present invention.

### BRIEF DESCRIPTION OF THE DRAWINGS

Various embodiments in accordance with the present disclosure will be described with reference to the drawings, in which:

FIG. 1 illustrates the formation of liposomes comprising DNA particles when DNA tags are mixed with Polysorbate 80 and HPMC under exemplary processes described herein.

FIG. 2 illustrates the formation of liposomes comprising DNA particles when DNA tags are mixed with stearic acid under exemplary processes described herein.

FIG. 3 illustrates the formation of liposome DNA particles when DNA tags are mixed with Polysorbate 80 under exemplary processes described herein.

FIG. 4 depicts a screen shot of a duplex Taqman TaqMan® qPCR assay. As depicted here, there is a clear differentiation in the amplification curves between the DNA tagged (+DNA) pills versus the non-DNA tagged (−DNA) pills after 35 cycles of qPCR (23 minutes). The software algorithm generates a simple yes/no output based on DNA barcode reading.

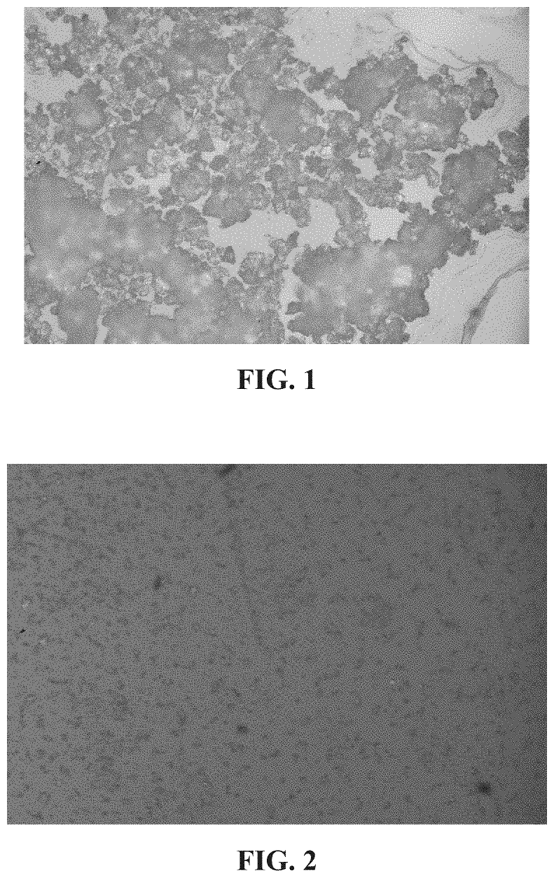

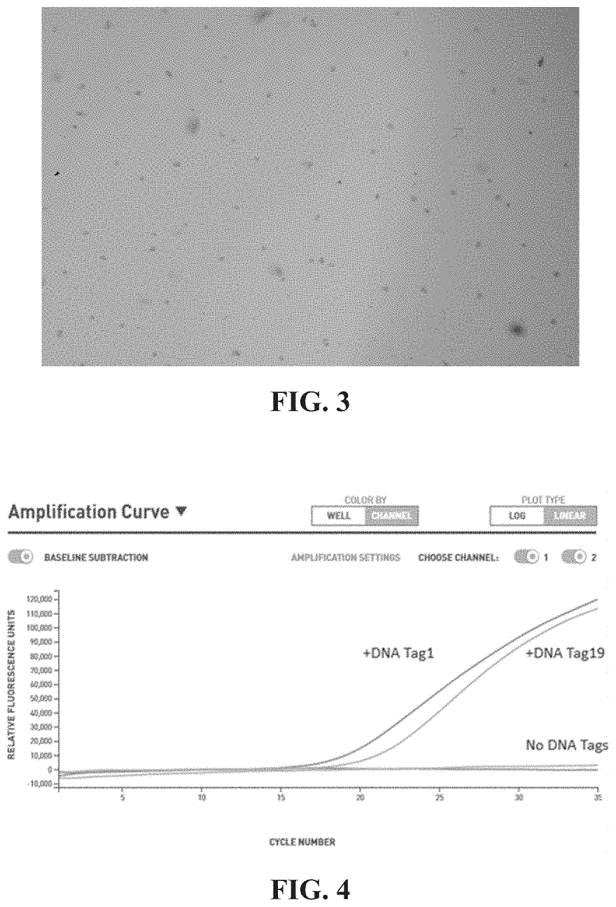

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

Modern commerce requires maximizing supply chain efficiencies, accountability, and security. The ability to track, and trace a product, and/or the components of a product, along its supply chain as well as throughout production and distribution, is becoming increasingly important and can help to address supply chain issues promptly.

This is relevant to a range of industries but is particularly important for global industries involving consumer products, including, for example, the pharmaceutical industry, the baby formula industry, the cosmetics industry, the vitamins and supplements industry, the nutraceuticals industry, the personal care industry, and others, which are becoming increasingly complex and widespread with respect to the origins and various locations a product and/or the components of a product pass through en route to an end user. This can include the manufacturers associated with one or more components of an ingredient/component, any associated processing and/or packing locations, any associated modes transportation, and any distribution outlets. Contamination and/or tampering can occur at any one or more of these points, or nodes, of the lifecycle of the product.

The ability to track, and trace a product, and/or the components of a product, allows possible problems with respect to the integrity of a product, e.g. contamination, to be identified and can allow for the identification of the origin of the problem. Such technology can help minimize the occurrence of material fraud, contamination, related illnesses, and their associated costs, including recall costs, legal expenses, and associated business losses.

Currently, product tracing technologies involve numerous stations along the supply chain, from producer to packer, distributor, retailer and ultimately the consumer. Product identification is generally applied to the packaging, rather than to the product itself. However, product packaging is frequently discarded and additionally does not provide sufficient information to identify the exact source of a problem that has been introduced at some point along the production, supply, or distribution chain. In most instances when a consumer detects product contamination and/or symptoms of illness, it may be days or weeks after the ingestion/consumption date. It is therefore desirable to provide an improved system and method for tracing products throughout the supply chain. Further, labeling a product with a tag that identifies two or more of the attributes of one or more of the nodes in the lifecycle of the product allows for easier, more efficient, and more accurate tracing of any possible sources of product contamination and tampering.

DNA tags have been explored as an efficient, effective and low cost food tracing system, as disclosed in, for example, U.S. Pat. No. 8,293,535, U.S. Application 2014/0057276, and U.S. Application 2014/0272097. However, such technologies do not allow for tracing product components to their origin.

There is an industry need for tracking and tracing via biologically benign or inert surrogates. In the pharmaceutical industry in particular, such media will necessarily require consumer-safe materials.

DNA tags can be used to trace food along multiple steps in the supply chain and ultimately to the consumer, by applying the product identification directly onto food products, at low cost, in a medium containing food-based FDA-approved sugars and a unique non-biological DNA tag. The resulting microparticle can be sprayed directly onto the product or mixed with a coating and will adhere to produce and other food surfaces. A practically limitless number of tags are possible by using synthetic or naturally occurring DNA.

Further detail regarding DNA tagging technology relevant to the embodiments described herein is provided in: International Patent Application No. PCT/US2016/038083, Pathogen Surrogates Based on Encapsulated Tagged DNA For Verification of Sanitation and Wash Water Systems for Fresh Produce; International Patent Application No. PCT/US2015/028880, DNA Based Bar Code for Improved Food Traceability; International Patent Application No. PCT/US2019/013069, Dispensing System for Applying DNA Taggants Used in Combinations to Tag Articles; International Patent Application No. PCT/US2019/017123, Source and Sanitation Assurance Testing of Foodstuffs and Sensitive Applications; International Patent Application No. PCT/US2019/029002, Sanitation Monitoring System Using Pathogen Surrogates and Surrogate Tracking; and U.S. Provisional Patent Application No. 62/723,974, Product Tracking and Rating System Using DNA Tags, which are incorporated herein by reference in their entirety and for all purposes.

The present disclosure encompasses techniques described and suggested herein which include forming binary (or non-binary) sequences that are encoded by the presence and/or absence of tags each comprising non-coding DNA snippets.

In order to better simulate a range of pathogenic substances (such as prions, viroids, viruses, and the like), a need exists for particles in the low-nanometer range, which can range in size from below 10 nanometers and greater. There is a particular need for the ability to achieve surface attachment of nano-tags with little change in aerosol or liquid dispersion characteristics.

Current tagging technology relies upon coating or attaching tags carried by molecular carriers onto commercial products. Various platforms can be used to deliver these molecular carriers. These include, for example, gelatins, proteins, saccharides, dextrins, poly-lactides, etc., and the like. However, such materials tend to exhibit aggregation either while in storage or upon dispersion. Such aggregation greatly reduces the surface area coverage by the molecular carriers. This leads to inefficient tagging and increased costs.

The present disclosure encompasses methods of generating a set of taggants to apply to a product (e.g. a pharmaceutical product), for example by using miniDART®, and tagging pharmaceutical products using the selected set of taggants (the set might represent a 32-bit “DNA barcode”). The taggants can be encapsulated by identifying one or more components of a coating, in the case of a pill or tablet as used in pharmaceutical products, vitamins or supplements, nutraceutical products, and the like, or one or more components of a product matrix or formulation, as in the case of pharmaceutical formulations, baby formula, cosmetics products, formulations for vitamins or supplements, formulations for nutraceutical products, personal care products, such as cream or lotions, and the like. The DNA taggants are then mixed with the identified components to form liposomal or micellar particles/structures. The tagged liposomal or micellar particles/structures are then added back to the original components of the coating or product matrix/formulation, at a concentration of about 1% or less, to manufacture the final consumer product (e.g. the pill or tablet as used in pharmaceutical products, vitamins or supplements, nutraceutical products, and the like, or the product matrix or formulation, as in the case of pharmaceutical formulations, baby formula, cosmetics products, formulations for vitamins or supplements, formulations for nutraceutical products, personal care products, such as cream or lotions, and the like).

In certain embodiments, liposomes can be formed by using the present DNA carrier as the lipophilic component, having an orientation which is directed by the surfactant package. Components such as, for example, natural oils, longer-chain glycerides, paraffins, and the like, can be infused with DNA, and the final structure can be verified through solvent washing and testing of residues. One skilled in the art would appreciate which types of components could be used as a lipophilic and/or surfactant component in accordance with some embodiments.

For cosmetics and personal care products, such formulations commonly include lipophilic components, such as, for example, essential oils, waxes, mono-tri-glycerides, fatty acids, and the like. For example, 18-carbon fatty acids are very common due to their skin compatibility and low cost. One skilled in the art would appreciate which types of components of a particular product, or type of product, could be used as a lipophilic and/or surfactant component in cosmetics or personal care product in accordance with the some embodiments.

Surfactants range from the synthetic Tween range to Spans to plant-based phosphatidylcholine.

Edibles such as baby formulas can be formed as Pickering emulsions with liposomal inclusions during the homogenization process of proteins, such as, for example, milk-, soy- and nut fruit-isolates, and the like. One skilled in the art would appreciate which types of components of a particular product, or type of product, could be used as a lipophilic and/or surfactant component in a baby formula preparation in accordance with the some embodiments.

Further information on liposome formation can be found at Andreas Wagner and Karola Vorauer-Uhl, J Drug Deliv. 2011, 591325, “Liposome Technology for Industrial Purposes”; and Akbarzadeh et al., Nanoscale Res Lett. 2013, 8:102, “Liposome: classification, preparation, and applications”; these references are incorporated herein by reference in their entirety and for all purposes.

Once robust component with a strong affinity to DNA are identified, such as an existing component from the cosmetics or personal care industries for a cosmetic or personal care product, the raw materials can be formed as liposomes and/or micelles under energetic conditions (e.g. stirring/homogenization/sonication/microfluidics).

Standard techniques for liposomal formation include, but are not limited to, the following:

High Energy: Industry standard.
Homogenization: Uses rotor stator and screen appropriate to the viscosity, usually at 2,000-12,000 rpm.
Sonication: Uses vibrational collision due to cavitation at transducer surface.
Microfluidics: Uses a pulsed energetic stream into a fusion cell, usually at 6,000-40,000 psi.

Liposomal and micellar structures are commonly employed to efficiently convey active components/ingredients (“actives”) within consumer products in the pharmaceutical, nutraceutical, food, personal care, baby formula, and cosmetics industries. Phospholipids can be used to form ordered spheres or solid-liquid architectures, which display an electrically-charged orientation, which contains the desired active. For example, soybean phosphatidylcholine is a commonly used phospholipid in the industry and among those skilled in the art; those skilled in the art will appreciate that additional phospholipids can be used and are encompassed within the context of the embodiments described herein. Within the presently described solutions containing active(s) and DNA taggants, grafts and couplings can additionally be incorporated.

The methods described herein can be used to create taggant liposomal or micellar structures/particles, which can enhance the binding of taggants to pill/tablet products or to formulary compounds. Taggant particles as described above can easily be removed from equipment surfaces by a standard sanitation procedure. The encapsulated taggants can include fluorescing compositions for quickly monitoring the presence or non-presence of taggants. The fluorescing compositions are stable, do not leave a mark, are non-visible under normal light, and fluoresce only under UV light. The fluorescing dye can be optical brightener. In an exemplary embodiment, a coating solution is prepared, to which single stranded DNA is added. The coating solution is then applied to, for example, a tablet including a pharmaceutical composition with one or more active ingredients. The recovery of DNA can be tested/verified, as well as the effectiveness of a cleaning/sanitation procedure in the removal of DNA taggants.

Coating Composition. The coating can be prepared from the following ingredients: a. EUDRAGIT® NM 30 D; b. HPMC (Vivapharm 5); c. Polysorbate 80 (33% aqueous sol.); d. Talc; and e. Water. For example, in an exemplary embodiment in accordance with some embodiments, to prepare 1 kg of coating material, the following specific amounts of each component can be used: a. EUDRAGIT® NM 30 D: 300 g weighed, 100 g dry; b. HPMC (Vivapharm 5): 10 g weighed, 10 g dry; e. Polysorbate 80 (33% aqueous sol.): 30.3 g weighed, 10 g dry; d. Talc: 100 g weighed, 100 g dry; e. Water: 559.7 g weighed. In another exemplary embodiment, to prepare 1 kg of coating material, the following specific amounts of each component can be used: a. Eudraguard protect 75 g; b. Steraric acid 7.5 g; c. Tartaric acid 5.4 g; d. Talc 75 g; e. Titanium dioxide 0-25 g; f. Demineralized water; g. 20% solids. In another exemplary embodiment, to prepare 1 kg of coating material, the following specific amounts of each component can be used: a. Eudraguard biotic 595.3 g; b. Polysorbat 80 10.8 g; c. Triethyl citrate 8.9 g; d. Glycerol mono Stearate 8.9 g; e. Water 376 g. One skilled in the art will appreciate that different components can be used and in varying quantities and ratios.

DNA Sequences. A set of taggants is generated to apply to a product using miniDART®, and products (e.g. pharmaceutical tablets) are tagged using the selected set of taggants; for example, the set might represent a 32-bit “DNA barcode”.

Taggants can be encapsulated by mixing taggants with carrier gel-phase HPMC and polysorbate 80, a surfactant.

The encapsulated taggants from are then mixed with EUDRAGIT 30D, HPMC, Polysorbate 80, and talc to generate the DNA-tagged coating solution for spraying onto the pills.

Single stranded DNA can be added to the coating solution described above. For example, 10-16 sequences generated from miniDART® can be tested. All DNA sequences can be added at the same time to the coating solution. The preparation can be divided into one or more portions, with one or more containing the DNA molecules, then the portions can be combined together to achieve the target coating composition.

Tablet Description. The coating solution containing DNA sequences, as described above, can be applied to a tablet; this can include, for example, a pharmaceutical composition with one or more active ingredients. In exemplary embodiments, the tablet can be selected from, for example, SafeWay Select, Acetaminophen, 500 mg; Walmart Equate, Aspirin, 325 m; and the like. One skilled in the art will appreciate that the DNA taggants described herein can be used with a wide range of products, such as pharmaceutical products, baby formula, cosmetics products, vitamins, supplements, nutraceuticals, personal care products, or any combination thereof.

Coating Application Method. The coating can be applied to a table using standard spray coating equipment, as would be appreciated by those skilled in the art.

DNA Concentration. DNA can be applied to the table at a concentration of 1 pg/tablet. 1 kg requires 2800 ng contained in 1 ml, to achieve the concentration of 1 pg/tablet.

Tablet Sampling Method. DNA can be sampled by rinsing with TE in a disposable tube. For example, one pill can be transferred using clean forceps into a 1.5-mL Eppendorf tube. A volume of 500 μL 1×TE is pipetted in. The tube is vortexed at max speed for approximately 2-3 seconds. A sample of the solution is then immediately pipetted into a separate 1.5-mL Eppendorf tube. The sample is then tested without further dilution.

DNA Cycle Quantification. The Ct can then be measured by Chai equipment, following standard procedure.

For any type of pills/tablets, the recovery/detection steps will be the same or very similar, regardless of whether the DNA taggants are added to the coating solution or directly to the active ingredients. For baby formula and cosmetic products, the recovery/detection steps can be very similar. In general, the tagged DNA can be recovered and detected from the tagged consumer products using any DNA extracting kit, such as, for example, the Macherey Nagel nucleospin DNA kit, and the like.

### Example 1 Below Describes an Exemplary Procedure in Accordance with Some Embodiments

### Exemplary Implementation #1

As described herein, the DNA taggants can be used in pharmaceutical, nutraceutical, vitamin, or supplement products. An exemplary procedure for use of the DNA taggants in a pill or tablet which can be used in these contexts is as follows.

DNA taggant particles are generated by mixing 0.1 g HPMC (Vivapharm 5), 0.303 g Polysorbate 80, 2800 ng DNA Tags in 25 mL water under homogenization (usually 2,000-12,000 rpm), with a rotor stator and screen appropriate to the viscosity. One skilled in the art will appreciate that other methods can be used to achieve the presently described liposomal/micellar structure, such as, for example, sonication, resulting in vibrational collision due to cavitation at the transducer surface, or microfluidics, via a pulsed energetic stream into a fusion cell, generally at 6,000-40,000 psi.

This DNA taggant-containing solution is then added into 975 mL coating solution containing EUDRAGIT® NM 30 D: 300 g weighed (100 g dry), HPMC (Vivapharm 5) 9.9 g weighed, Polysorbate 80 (33% aqueous sol.) 29.997 g weighed, 9.9 g dry, Talc: 100 g weighed, 100 g dry.

This tagged coating solution is then used to spray pills (e.g. SafeWay Select, Acetaminophen, 500 mg), according to the following steps:

1) Apply one full spray onto 100×500 ug Pills, let them air dry 5-10 min;
2) Turn all pills halfway (180 degrees);
3) Spray one more time onto 100×500 ug Pills, let them air dry 5-10 min.

The tagged pills are then subject to standard qPCR detection as follows:

1) Remove appropriate number of qPCR test strips from −20° C.
2) Allow test strips to equilibrate to room temperature (20-25° C.) before use, approximately 10 minutes.
3) Spin down test strips using the included microcentrifuge, max speed for 3 s.
4) Place strips and swabs into sample collection holder, in appropriate order.
5) Remove film from test kit strip.
6) Transfer one tagged pill using clean forceps into a 1.5-mL Eppendorf tube. Pipet in 500 μL 1×TE. Vortex the tube at max speed for approximately 2-3 seconds.
7) Dispense the 2 μL sample into the appropriate corresponding well of the test strip.
8) Remove one test strip cap from package and firmly place onto the test strip.
9) Spin down test strip using the included microcentrifuge, max speed for 3 s.
10) Load test strips into CHAI qPCR instrument and initiate qPCR reading, as per operating instruction in manufacturer's manual for 35 cycles. Results will be displayed in Ct values following qPCR reading, approximately 23 minutes after qPCR initiation.

TABLE 1
Ct read from Tagged pills using tagged coating solution

| | | SEQ. 1 | | SEQ. 19 |
|---|---|---|---|---|
| DNA | Number of | Average | Number of | Average |
| applied | replicates | Ct on Pill | replicates | Ct on Pill |
| 0 | 10 | 34.78 | 10 | 34.71 |
| 2.8 | 10 | 21.9 | 10 | 23.6 |
| ng/mL | | | | |

Smaller particles produce a greater surface area per given unit mass. Using greater surface area translates into an increased efficiency of dispersion for the DNA taggants carried by the particles. As such, particle sizing should be closely controlled for cost-effective dose planning.

This technology relies upon the discrete molecular forms displayed in, for example, colloids, Pickering emulsions, liposomes and nanosomes, etc., and the like, ranging in particle size from tens of microns down to 100 nanometers, and below.

In some embodiments, EUDRAGIT® NM 30 D, HPMC (Vivapharm 5), Polysorbate 80 (33% aqueous sol.), talc, and water are used in the following ratios to prepare 1 kg of coating:
EUDRAGIT® NM 30 D: 300 g weighed; 100 g dry.
HPMC (Vivapharm 5): 10 g weighed; 10 g dry.
Polysorbate 80 (33% aqueous sol.): 30.3 g weighed; 10 g dry.
Talc: 100 g weighed; 100 g dry.
Water: 559.7 g weighed.

In some embodiments, the product is prepared as a spray-dried product. It can be prepared in deionized or distilled water with enough water molecules to allow complete self-assembly of the particles.

One skilled in the art will appreciate that the above-described experimental procedures are exemplary. One skilled in the art will further understand where modification of the above-described process can achieve comparable results, i.e. formation of a pill/tablet coating, into which DNA taggants can be incorporated. One skilled in the art will further be able to determine varying concentrations of the components used in formation of the pill/tablet coating.

In the exemplary embodiment described above, the coating comprises a concentration of 1% or less of DNA taggants in a consumer-safe solution. Embodiments encompass coatings and product formulations comprising DNA taggant concentrations ranging from less than about 1%, less than about 0.90%, less than about 0.80%, less than about 0.70%, less than about 0.60%, less than about 0.50%, less than about 0.40%, less than about 0.30%, less than about 0.20%, less than about 0.10%, less than about 0.05%, or less than about 0.02%, or any intermediate values or ranges derived therefrom, in a consumer-safe carrier solution. For example, embodiments encompass coatings comprising DNA taggant concentrations of less than about 0.001% to less than about 1% in a consumer-safe carrier solution. For example, the US Food and Drug Administration (FDA) have recommended upper limits of DNA concentrations of 10 ng/dose and 200 base pairs. Embodiments encompass coatings and product formulations comprising DNA taggant concentrations ranging from 0.01 pg per pill, 0.05 pg per pill, 0.10 pg per pill, 0.50 pg per pill, 0.60 pg per pill, 0.70 pg per pill, 0.80 pg per pill, 0.90 pg per pill, 1 pg per pill, 2 pg per pill, 3 pg per pill, 4 pg per pill, 5 pg per pill, 6 pg per pill, 7 pg per pill, 8 pg per pill, 9 pg per pill, 10 pg per pill, 11 pg per pill, 12 pg per pill, 13 pg per pill, 14 pg per pill, 15 pg per pill, 20 pg per pill, 30 pg per pill, 40 pg per pill, 50 pg per pill, 75 pg per pill, 100 pg per pill, and greater, or any intermediate ranges or values derived therefrom.

In some embodiments, the DNA taggant concentrations range from about 1 pg per pill to about 10 pg per pill.

### Taggant Complex

This complex of DNA tags with pill/tablet coating solutions forms a robust, stable carrier system with an affinity for hard, smooth and/or porous surfaces. This complex further allows for facile harvesting of taggant through swabbing or immersion in recovery reagent chemistries. This complex further allows for its use as a polymer component to existing consumer-safe applications.

Accordingly, as described herein, this technology allows for the use of DNA taggants in consumer product coatings. Alternatively, the DNA taggants can be mixed directly into the product formulation.

Further, this functional coupling can act as a drop-in plasticizer to edible film-forming starches, carbohydrates, and other natural or synthetic ingredients capable of polymerizing with propanediol. This is beneficial because in many practical formulations of films and coatings, water, alcohol, and water/alcohol solutions are not suitable, as they evolve from the formulation during the heating and curing phases, leaving bubbles, air passages and weak stress areas in the product morphology.

Exemplary film and coating materials include, for example, natural polymers and synthetic polymers, such as those listed below.

Natural polymers applicable include, but are not limited to, the following: pullulan, starch, gelatin, pectin, sodium alginate, maltodextrin, polymerized rosin, and the like; synthetic polymers applicable include, but are not limited to, the following: hydroxy propyl methyl cellulose, sodium carboxy methyl cellulose, poly ethylene oxide, hydroxy propyl cellulose, poly vinyl pyrrolidone, poly vinyl alcohol, and the like.

As such, according to some embodiments, films, coatings, reinforcing polymers, etc., and the like, can be encoded as part of a comprehensive method of tracking and traceability. This can include DNA tags within and atop the product as well as the packaging materials. The set of non-coding DNA sequences used in the taggant forms a data element that can be represented by a binary word.

The existence and tracking of specific binary words can be combined with the use of public blockchains so that a relationship between a source, the binary word, the consumer product and other relationships can be publicly posted and be unalterable. Item level traceability enables swift response to outbreaks, counterfeiting, adulteration, etc. and with this information posted to a public blockchain, it can be traced and responded to by others unrelated to the provider of the consumer product. This item level traceability can also be a key to fulfilling sustainability and responsible sourcing promises to consumers, as well as reducing human and economic impact of outbreaks and recalls.

Techniques described and suggested herein include methods and apparatus to apply DNA taggants to smaller batches of material from different producers before the smaller lots are aggregated into larger batches or shipments, allowing the producer of a particular unit of the material to be identified even after the smaller lots have been aggregated.

More generally, a DNA taggant set may be applied to a small lot of product. After the DNA taggant set is applied to the small lot of product, the small lot of product may be combined with other small lots to form a larger aggregated batch of product. During processing, individual units or portions of the product may be sampled or inspected. The grading may be binary (e.g., acceptable or unacceptable) or may be more fine grained (e.g., unacceptable, poor, good, excellent, or grading on a scale). The grading may be done by attaching a grading label to a sample (e.g., adhesively or mechanically), by placing the sample in a labelled receptacle, or by marking the sample (e.g., with ink or paint). The graded sample or attachment may be analyzed to determine the DNA taggant set, which in turn correlates to the tag string, which in turn correlates to the producer or lot, allowing the grade label from the label, receptacle, or mark to be associated with the producer or lot.

In another embodiment, the same DNA taggant combination may be used for a larger lot or to identify producers, and the DNA taggant combination may be changed infrequently (e.g., a few times a day). The taggant combination may be distributed from a premixed taggant combination or may be mixed in a sprayer's manifold to produce the required taggant combination.

DNA taggants correspond to encoded information that can be applied to objects, including consumer products, in a manner that allows for later reading of this encoded information from the objects. In a specific embodiment, the DNA taggants are unique, each taggant represents a bit position and the pattern of presence or absence of one of the DNA taggants corresponds to a bit value of 1 or 0 and the pattern of DNA taggants that are present or absent forms a binary number representing the encoded information. In another embodiment, the presence of a first DNA taggant is used to signal a value of “1” of the encoded information and the presence of a second DNA taggant is used to signal a value of “0” of the encoded information. The apparatus that adds the DNA taggants to the objects can be configured such that the encoded information can change from object to object and not cross-contaminate objects with DNA taggants that are for one object but not another.

### Pill/Tablet Formulations

The consumer products including coatings containing DNA taggants, as disclosed herein, can be prepared and administered in a wide variety of oral, parenteral, and topical dosage forms. Preferred embodiments of the methods described herein involve oral administration of one or more compounds described herein. The consumer products described herein can additionally be administered by injection (e.g. intravenously, intramuscularly, intracutaneously, subcutaneously, intraduodenally, or intraperitoneally). Also, the compounds described herein can be administered by inhalation, for example, intranasally. Additionally, the consumer products disclosed herein can be administered transdermally. It is also envisioned that multiple routes of administration (e.g., intramuscular, oral, etc.) can be used to administer the consumer products disclosed herein.

Consumer products, such as pharmaceutical products, nutraceuticals, health supplements, and vitamins can be administered in solid or liquid form as appropriate with the desired method of administration. For oral administration, the consumer product can be administered as a solid or a liquid for in various embodiments. For some embodiments that are administered as injections, the consumer product can be delivered as a liquid or in a liquid suspension.

In some oral embodiments, the consumer products disclosed herein can be administered as solids, more specifically in the form of tablets, lozenges, troches, powders, granules, or capsules. In some other oral embodiments, the consumer products disclosed herein can be administered as liquids more specifically as solutions, aqueous or oily suspensions, capsules, emulsions, syrups or elixirs. The composition for oral use can contain one or more agents selected from the group of sweetening agents, flavoring agents, coloring agents and preserving agents in order to produce elegant and palatable preparations. Accordingly, there are also provided compositions comprising a consumer acceptable carrier or excipient and one or more compounds disclosed herein.

In powders, the carrier is a finely divided solid in a mixture with the finely divided active component. In tablets, the active component is mixed with the carrier having the necessary binding properties in suitable proportions and compacted in the shape and size desired. Suitable carriers are magnesium carbonate, magnesium stearate, talc, sugar, lactose, pectin, dextrin, starch, gelatin, tragacanth, methylcellulose, sodium carboxymethylcellulose, microcrystalline cellulose, mannitol, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included.

In some embodiments, tablets contain an active ingredient in admixture with non-toxic acceptable excipients that are suitable for the manufacture of tablets. These excipients can be, for example, (1) inert diluents, such as calcium carbonate, lactose, calcium phosphate, carboxymethylcellulose, or sodium phosphate; (2) granulating and disintegrating agents, such as corn starch, alginic acid, and polymers such as Kollidon® CL, also known as crospovidone; (3) binding agents, such as starch, gelatin or acacia; and (4) lubricating agents, such as magnesium stearate, stearic acid or talc. These tablets can be uncoated or coated with a film or layer by known techniques to delay disintegration and absorption in the gastrointestinal tract and thereby provide a sustained action over a longer period. For example, a time delay material such as glyceryl monostearate or glyceryl distearate can be employed. Other examples include Eudragit® L 30 D-55, a polymer that includes copolymerized methacrylate and which prevents dissolution below pH 5.5.

In some embodiments, tablets contain the active ingredient as an amorphous solid. This can be achieved by generating an amorphous solid dispersion containing the active ingredient and at least one polymer. In some embodiments, that polymer is Kollidon® VA64 which is a vinylpyrrolidone-vinyl acetate copolymer. One of skill in the art will appreciate that a certain weight ratio of active ingredient to polymer is necessary to maintain the active ingredient in an amorphous state. This active ingredient:polymer weight ratio can range from 1:1 to upwards of 1:10. In some embodiments, an active ingredient:polymer weight ratio is 1:3. One of skill in the art will also appreciate that there are a variety of techniques available to produce an amorphous solid dispersion including holt melt extrusion and spray-dried dispersion (SDD) methods.

In certain embodiments, it can be desirable to control particle size distribution. A number of techniques, including micronization techniques, can be employed to produce a desired particle size distribution in certain embodiments of a given formulation.

Also included are solid form preparations that are intended to be converted, shortly before use, to liquid form preparations for oral administration. Such liquid forms include solutions, suspensions, and emulsions. These solutions can be of water or water/propylene glycol mixtures. These preparations can contain, in addition to the active component, one or more colorants, flavors, stabilizers, buffers, artificial and natural sweeteners, surfactants, dispersants, thickeners, solubilizing agents, and the like.

Aqueous solutions suitable for oral use can be prepared by dissolving the consumer products containing an active component in water and adding suitable colorants, flavors, stabilizers, and thickening agents as desired. Aqueous suspensions suitable for oral use can be made by dispersing the finely divided active component in water with viscous material such as natural or synthetic gums, resins, methylcellulose, sodium carboxymethylcellulose, and other well-known suspending agents.

When parenteral application is needed or desired, particularly suitable admixtures for the compounds disclosed herein are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, polyethylene glycol, and the like. Ampoules are convenient unit dosages. The compounds disclosed herein can also be incorporated into liposomes or administered via transdermal pumps or patches. Pharmaceutical admixtures suitable for use in the pharmaceutical compositions and methods disclosed herein include those described, for example, in PHARMACEUTICAL SCIENCES (17th Ed., Mack Pub. Co., Easton, Pa.) and WO 96/05409, the teachings of both of which are hereby incorporated by reference.

In some embodiments, preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles including fluid and nutrient replenishers, electrolyte replenishers (such as those based on Ringer's dextrose), and the like. Preservatives and other additives can also be present such as, for example, antimicrobials, anti-oxidants, chelating agents, growth factors and inert gases and the like.

Some consumer products can have limited solubility in water and therefore can require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions can be desirable to decrease variability in dispensing the formulations, to decrease physical separation of components of a suspension or emulsion of formulations, and/or otherwise to improve the formulation. Such viscosity binding agents include, for example, polyvinyl alcohol, polyvinyl pyrrolidone, methyl cellulose, hydroxypropyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

Aqueous suspensions normally contain the active materials in admixture with excipients suitable for the manufacture of the aqueous suspension. Such excipients can be (1) suspending agent such as sodium carboxymethyl cellulose, methyl cellulose, hydroxypropyl methylcellulose, sodium alginate, polyvinylpyrrolidone, gum tragacanth and gum acacia; (2) dispersing or wetting agents which can be (a) naturally occurring posphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

Preservatives include antimicrobial, anti-oxidants, chelating agents and inert gases. Other acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV., 14th ed. Washington: American Pharmaceutical Association (1975), the contents of which are hereby incorporated by reference. The pH and exact concentration of the various components of the composition are adjusted according to routine skills in the art. See e.g. Goodman and Gilman (eds.), 1990, THE PHARMACOLOGICAL BASIS FOR THERAPEUTICS (7th ed.).

A consumer product disclosed herein can also be administered in the form of suppositories for rectal administration of the drug. These compositions can be prepared by mixing the drug with a suitable non-irritating excipient that is solid at ordinary temperature but liquid at the rectal temperature and will therefore melt in the rectum to release the drug. Such materials include cocoa butter and polyethylene glycols.

For preparing suppositories, a low melting wax, such as a mixture of fatty acid glycerides or the aforementioned cocoa butter, is first melted and the active component is dispersed homogeneously therein, as by stirring. The molten homogeneous mixture is then poured into convenient sized molds, allowed to cool, and thereby to solidify.

For topical use, creams, ointments, jellies, solutions or suspensions, etc., containing the compounds disclosed herein are employed.

The consumer products disclosed herein as used in the methods disclosed herein can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

For in vivo application, a consumer product disclosed herein, can be administered parenterally by injection or gradual perfusion over time. Administration can be intravenously, intraperitoneally, intramuscularly, subcutaneously, intracavity, or transdermally. For in vitro studies the consumer products can be added or dissolved in an appropriate biologically acceptable buffer and added to a cell or tissue.

The pill/tabletpreparation is preferably in unit dosage form. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation can be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

The compositions described herein are preferably prepared and administered in dose units. For treatment of a subject, depending on activity of the compound, manner of administration, nature and severity of the disease or disorder, age and body weight of the subject, different daily doses can be used. Typically, dosages used in vitro can provide useful guidance in the amounts useful for in situ administration of the composition, and animal models can be used to determine effective dosages for treatment of particular disorders.

Under certain circumstances, however, higher or lower daily doses can be appropriate. The administration of the daily dose can be carried out both by single administration in the form of an individual dose unit or else several smaller dose units and also by multiple administrations of subdivided doses at specific intervals.

Various considerations are described e.g., in Langer, 1990, Science, 249:1527; Goodman and Gilman's (eds.), 1990, Id., each of which is herein incorporated by reference and for all purposes. Dosages' parenteral administration of active pharmaceutical agents can be converted into corresponding dosages for oral administration by multiplying parenteral dosages by appropriate conversion factors. As to general application, dosages from in vivo animal studies can be adapted to a human equivalent dose (HED) by applying the appropriate animal scale factor to the mg/kg ratio for the given in vivo animal. An average adult human weighs about 60 kg. See e,g, GUIDANCE FOR INDUSTRY: Estimating the Maximum Safe Starting Dose in Initial Clinical Trials for Therapeutics in Adult Healthy Volunteers (FDA Guidance; July 2005).

Embodiments also encompass tablets including a pharmaceutical composition. In some embodiments, the pharmaceutical composition can include one or more active ingredients. In some embodiments, the one or more active ingredients in the pharmaceutical composition can exist as an amorphous solid in an amorphous solid dispersion. In some embodiments, the amorphous solid dispersion can be 50% of the tablet by weight. In some embodiments, the amorphous solid dispersion includes a first polymer.

In some embodiments, the tablets can include at least one disintegrant. In some embodiments, the disintegrant includes crospovidone. In some embodiments, the tablets can include at least one filler. In some embodiments, the filler includes microcrystalline cellulose or mannitol. In some embodiments, the tablets can include at least one lubricant or glidant. In some embodiments, the lubricant or glidant includes magnesium stearate or talc.

In some embodiments, the tablets can include an exterior layer or film. In some embodiments, the exterior layer or film can include at least a second polymer. In some embodiments, the second polymer can prevent dissolution of the tablet below pH 5.5. In some embodiments, the second polymer can be Eudragit® L 30 D-55. In some embodiments, the second polymer is a methacrylic acid—ethyl acrylate copolymer.

In some embodiments, the tablets include an exterior layer of a second polymer, and wherein the tablet without said exterior layer can be 50% by weight the amorphous solid dispersion, 10% by weight crospovidone, 2% by weight magnesium stearate, 19% by weight microcrystalline cellulose, 18% by weight mannitol, and 1% by weight talc. In some embodiments, the second polymer can be Eudragit® L 30 D-55.

Embodiments described herein also encompass processes of manufacturing the aforementioned tablets, wherein the process can include: (1) producing an amorphous solid dispersion of the one or more active ingredients; (2) granulating said amorphous solid dispersion of step (1) with intragranular raw materials in dry conditions; (3) blending said granules of step (2) with extragranular raw materials to form a final mixture; (4) compressing said final mixture of step (3) into a tablet; and (5) coating said tablet of step (4) with a film or layer. In some embodiments, the process can further include: (1) producing an amorphous solid dispersion of the one or more active ingredients using a spray-dry dispersion (SDD) technique; (2) mixing said amorphous solid dispersion of step (1) with intragranular raw materials comprising at least one disintegrant and at least one lubricant; (3) dry granulating said mixture of step (2), wherein said granulation process comprises using a roller compactor to produce compacted ribbons, wherein said compacted ribbons are subsequently milled into granules; (4) blending the granules of step (3) with delumped extragranular raw materials comprising a disintegrant and a lubricant; (5) compressing the blend of step (4) into a tablet; and (6) coating said tablet of step (5) with a film or layer, where the film or layer additionally includes the presently described DNA taggants.

In further embodiments, the tablets are coated with an exterior film or layer called an enteric coating, where the enteric coating additionally includes the presently described DNA taggants. This layer, which comprises a polymer and/or other materials, can provide additional properties, such as resistance to dissolution in an environment below pH 5.5. In some embodiments, the polymer is a copolymer that comprises copolymerized methacrylate. One of skill in the art will appreciate that there are a variety of techniques available to coat the tablets with this exterior layer. In some embodiments a pan coating technique is employed.

### Pill/Tablet Coating Composition

As described above in Example 1, coating solutions have been prepared by mixing the presently described DNA taggants with EUDRAGIT® NM 30 D, HPMC (Vivapharm 5), Polysorbate 80 (33% aqueous sol.), Talc, and water. One skilled in the art can contemplate which other coating ingredients can be used in accordance with some embodiments, along with the relevant ratios of each component.

For example, enteric polymers can be applied to a tablet/pill in organic or aqueous solvents onto the unit dosage forms as solutions in organic or aqueous solvents, such as water, methylene chloride, ethanol, methanol, isopropyl alcohol, acetone, ethyl acetate and combinations thereof; one skilled in the art will appreciate which other solvents can be appropriate in the context of the embodiments described herein. The solvent is chosen based on the polymer solubility, ease of evaporation, and viscosity of the solution.

For example, some polymers are also available as aqueous systems, such as these Eudragit® L30D (methacrylic acid-ethyl acrylate ester copolymer, Rohm-Haas GmBH, West Germany); Aquateric® (cellulose acetate phthalate-containing polymer, FMC Corporation, Philadelphia, Pa.); and Coateric® (polyvinyl acetate phthalate-based product, Colorcon, Inc., West Point, Pa.). Aqueous-based systems can be prepared at high concentration without encountering high viscosity, and do not have the problems associated with organic systems, such as flammability, toxicity of residual solvent in the dosage form, etc.

Pills/tablets can be coated using methods known to those skilled in the art. These include, for example, fluidized bed equipment, perforated pans, pharmaceutical pans, compression coatings, continuous or short spray methods, or drenching. See also the procedure described in Example 1. In one embodiment, the solid unit dosage forms are coated by continuous spray methods. In one embodiment, the outer coating layer is applied after the inner coating layer but before the inner coating layer is dried and/or cured. In yet another embodiment, the outer coating layer is applied within seconds, after the inner coating layer is applied. If a shiny finish coat is desired on the solid dosage forms of some embodiments, a small quantity of polyethylene glycol can be applied to the finished dosage form.

In one embodiment, all of the dosage forms of some embodiments are uniform in size prior to coating with the coating layers. This allows for uniform coating thickness and more uniform dissolution of the coating layers.

### Pill/Tablet Dosages

Compositions provided herein include compositions wherein the active ingredient is contained in a therapeutically effective amount, i.e., in an amount effective to achieve its intended purpose. The actual amount effective for a particular application will depend, inter alia, on the condition being treated.

The dosage and frequency (single or multiple doses) of a consumer product to be consumed or administered can vary depending upon a variety of factors, including route of administration; size, age, sex, health, body weight, body mass index, and diet of the recipient; nature and extent of symptoms of the disease being treated (e.g., the disease responsive to inhibition of thrombin); presence of other diseases or other health-related problems; kind of concurrent treatment; and complications from any disease or treatment regimen. Other therapeutic regimens or agents can be used in conjunction with the methods and compounds disclosed herein.

For any consumer product described herein, the therapeutically effective amount can be initially determined from a variety of techniques known in the art, e.g., biochemical characterization of inhibition of thrombin, cell culture assays, and the like. Target concentrations of an active compound will be those concentrations of active compound(s) that are capable of decreasing enzymatic activity as measured, for example, using the methods described.

Therapeutically effective amounts for use in humans can be determined from animal models. For example, a dose for humans can be formulated to achieve a concentration that has been found to be effective in animals. The dosage in humans can be adjusted by monitoring enzymatic inhibition and adjusting the dosage upwards or downwards, as described above.

Dosages can be varied depending upon the requirements of the patient and the compound being employed. The dose administered to a patient, in the context of the methods disclosed herein, should be sufficient to affect a beneficial therapeutic response in the patient over time. The size of the dose also will be determined by the existence, nature and extent of any adverse side effects. Generally, treatment is initiated with smaller dosages, which are less than the optimum dose of the compound. Thereafter, the dosage is increased by small increments until the optimum effect under circumstances is reached. In some embodiments of a method disclosed herein, the dosage range is 0.001% to 10% w/v. In some embodiments the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

Accordingly, in some embodiments, dosage levels of the compounds disclosed herein as used in the present methods are of the order of, e.g., about 0.1 mg to about 1 mg, about 1 mg to about 10 mg, about 0.5 mg to about 20 mg per kilogram body weight, and average adult weighing 60 kilograms, with a preferred dosage range between about 0.1 mg to about 20 mg per kilogram body weight per day (from about 6.0 mg to about 1.2 g per patient per day). The amount of the compound disclosed herein that can be combined with the carrier materials to produce a single dosage will vary depending upon the host treated and the particular mode of administration. For example, a formulation intended for oral administration to humans can contain about 5 μg to 1 g of a compound disclosed herein with an appropriate and convenient amount of carrier material that can vary from 5 to 95 percent of the total composition. Dosage unit forms will generally contain between from about 0.1 mg to 500 mg of a compound disclosed herein.

It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy.

The ratio between toxicity and therapeutic effect for a particular compound is its therapeutic index and can be expressed as the ratio between LD50 (the amount of compound lethal in 50% of the population) and ED50 (the amount of compound effective in 50% of the population). Compounds that exhibit high therapeutic indices are preferred. Therapeutic index data obtained from in vitro assays, cell culture assays and/or animal studies can be used in formulating a range of dosages for use in humans. The dosage of such compounds preferably lies within a range of plasma concentrations that include the ED50 with little or no toxicity. The dosage can vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration, and dosage can be chosen by the individual practitioner in view of the patient's condition and the particular method in which the compound is used. For in vitro formulations, the exact formulation and dosage can be chosen by the individual practitioner in view of the patient's condition and the particular method in which the compound is used.

Having described some embodiments in detail, it will be apparent that modifications, variations, and equivalent embodiments are possible without departing from the scope of the invention defined in the appended claims. Furthermore, it should be appreciated that all examples in the present disclosure are provided as non-limiting examples.

### Exemplary Implementation #2

As described herein, the DNA taggants can be used in pharmaceutical, nutraceutical, vitamin, or supplement products. An exemplary procedure for use of the DNA taggants in the formulation itself, such as in combination with one or more active ingredients in the formulation, which can be used in these contexts is as follows. The DNA taggants described herein can be added to either the formulation containing the active ingredient, or to the coating solution, or to both the formulation containing the active ingredient and the coating solution.

A hydroxytyrosol active ingredient is tagged with DNA in the following procedure:

(1) Part 1: Under high shear homogenization, a 6-7-8 carbon cyclic oligosaccharide is added to 18 megohm water.
(2) Part 2: Into this stock solution, and under inert atmospheric conditions, is added a pharmaceutical oleuroprene derivative, hydroxytyrosol at a 2:1-4:1 molar concentration.
(3) Part 3: Under microfluidic cycling, the DNA taggant is insinuated prior to the fusion cell for the number of cycles necessary to form a complete complexation reaction. The final architecture is a Schardinger inclusion complex containing the active drug and DNA within the cyclic cage architecture of the new molecule. This active ingredient package is now prepared either as a liquid gel-cap serum or lyophilized under inert atmosphere for anhydrous dosing.
(4) Different DNA taggants can optionally be subsequently incorporated into the coating solution or the capsule chemistry, as discussed earlier. This allows for binary tagging of tablets, caplets and capsules.
(5) The tagged product can be extracted using any DNA extract kit, such as Macherey Nagel nucleospin DNA kit.
(6) DNA taggant can be detected by applying above extract to qPCR and running qPCR on Chai for 35 cycles.

### Exemplary Implementation #3

As described herein, the DNA taggants can be used in cosmetics and personal care products. An exemplary procedure for formation of the liposome phase for a humectant is as follows:

1. (Part 1) A medium-chain triglyceride such as Stepan Neobee 5 is blended with the DNA taggant under moderate stirring for five minutes.
2. (Part 2) A surfactant, such as, for example, phosphatidylcholine (Avanti Polar), in a ratio of 40% to 60% of MCT from Part 1 is introduced to the deionized water bath under stirring for five minutes.
3. Part 1 is insinuated into Part 2 under homogenization using a Silverson LHT system in an open beaker. Phase formation occurs over ten minutes at 6,000 rpm.
4. The final liposomal emulsion is set aside or optionally placed in vacuum for degassing.
5. This formula is now available and can be incorporated into various cosmetics or personal care products, such as skin serums, creams, lotions, colorway, foundation, makeup, etc.
6. Tagged cosmetic product can be extracted using any DNA extract kit, such as Macherey Nagel nucleospin DNA kit.
7. DNA taggant can be detected by applying above extract to qPCR and running qPCR on Chai for 35 cycles.

### Exemplary Implementation #4

As described herein, the DNA taggants can be used in baby formula or protein dairy formula products (e.g. FDA 121.11 Medical Food). An exemplary procedure for formation of the liposome phase for a humectant is as follows:

1. (Part 1.) A milk protein isolate, such as, for example, Idaho Milk Products “IdaPro milk protein isolate” is blended with DNA taggants under mild stirring in deionized water for 20 minutes.
2. (Part 2.) Soy phosphatidylcholine as soy lecithin liquid (IFC Solutions) in a relative percentage of 40% to 60% of the protein isolate mass is solubilized under moderate shear with the final bulk quantity of deionized water for five minutes.
3. Parts 1 and 2 are introduced simultaneously via a twin-feed mechanism into a microfluidizer (Microfluidics Model 110F) at 10,000 psi using a single-pass recovery.
4. The final liposomal product is set aside or optionally degassed under vacuum. This formula is now available for incorporation into baby formula or a protein dairy formula.
5. Tagged baby formula products can be extracted using any DNA extracting kit, such as, for example, Macherey Nagel nucleospin DNA kit.
6. DNA taggant can be detected by applying above extract to qPCR and running qPCR on Chai for 35 cycles.

### Exemplary Implementation #5

In a specific example, there is a set of 32 DNA taggants to work with and so there are 232 possible combinations of DNA taggants that can be applied to an object, thus encoding the object with a 32-bit value corresponding to a specific “tag string” that might be represented by a sequence of 32 binary values each having a bit location in the tag string. It should be understood that other numbers are also possible and in the general case, a tag string might be represented as an indexed array of values, each having an index or position in the tag string, where the values might be binary values.

For example, it might be that 28 bits (and 28 DNA taggants) would be sufficient for a particular application. For example, if there are 128 producers of apples, each having 8 facilities, and they group their apples by lot such that they output 256 lots per year, one per day, over the course of 16 years, the manufacturer, facility, lot, and year can be encoded in a 7+3+8+4=22 bit tag string and so 22 bits and 22 DNA taggants are sufficient and that leaves room for checksum bits/taggants to be added. In this simplified example, the number of possible values for each of the variables is a power of two, but that is not required and other values can be used. Conventional mapping of values to tag strings can be done.

As used herein, a DNA taggant is a material that includes an oligonucleotide and possibly other material. In a specific example, each DNA taggant comprises a static part and an identifier part, wherein all of the DNA taggants have the same static part and thus it can be used to differentiate between the set of DNA taggants in use and other DNA that might be present in a sample. Preferably, the presence of a DNA taggant can be done even when there are very low concentrations of the DNA taggant in or on the object. Thus, where there is a dispersant expected to be found in the sample, the sample and/or the dispersant thereon is sampled, detected, error-corrected as needed, etc. to determine the tag string that was applied to the object.

A tag string has an associated DNA taggant set (sometimes called a “DNA barcode”), which is a selection of particular DNA taggants used, or to be used, on an object to “label” that object with the tag string. The object might be an item being sold, bulk material, packaging, or other physical object or item where labeling according to the tag string is useful. In particular, where a printed label is not workable or viable, applying the tag string could be done instead. For illustration purposes, consider the case where the tag string comprises binary values each having a bit position in the string, such as “01101001 10101001 10100111 10010101” which has a “0” in the first bit position, “1” in the second and third bit positions, and “1” in the 32nd bit position. A specific DNA taggant is associated with each bit position of the tag string and labeling an object might comprise determining which bit positions of the tag string are “1”, determining which DNA taggants go with those bit positions, and applying those DNA taggants (referred to herein as a “taggant set”) to the object, and not applying the DNA taggants that go with the bit positions of the tag string that have “0” values. Alternatively, pairs of DNA taggants might be used, wherein the presence of one taggant indicates a “1” in a position and the presence of the other indicates a “0” in that position. Though this approach uses twice as many taggants for the same number of bits of information, it provides error checking. Other approaches used error checking codes are possible.

The tag string could represent different information. For example, in a particular industry or application, some of the bit positions might correspond to the company name, others to a serial number, others to a production date or location, etc. By later sampling the object on which the taggant set was applied, a tag detecting system can decode the taggant set and from there determine the tag string that was applied to the object.

In some embodiments, all or part of the tag string is an index value that points to a record in an external database that provides data about that particular record. In those embodiments, the tag string assigned to an object might be entirely arbitrary and an external database of object information would be used to get data about the object rather than decoding any data about the object from the bit pattern itself.

In an example distribution system, there are lots and each lot has applied to it a specific tag string and a first lot receives a first taggant set corresponding to a first tag string and then a second lot receives a second taggant set corresponding to a second tag string different than the first lot. The first lot might be multiple items, such as a plurality of melons, or the first lot might be a single item, such as a bag of coffee beans. In the case of a bag, the distribution system might be integrated in with an automated bag filling line. In such a line, a new bag is positioned in the system and is clamped to the filling line chute to receive product. Perhaps before the first bag is in place, the distribution system initially dispenses plain carrier (no taggants) in the “dead volume” (the volume of the piping beyond actuating valves). Then when the empty bag is in place, or after the bag is filled, but before it is declamped and stitched closed, the distribution system actuates certain valves of the distribution system to push out the plain carrier and then push out specific taggants based on that bag's designated tag string. It may be that delivery is timed so that the plain carrier residing in the dead volume is delivered during clamping to the bottom of the empty bag and the taggants are delivered as the product is filling the bag. The taggant valves may be de-energized before the bag is full while the plain carrier valve remains energized, so that at the completion of the bag filing cycle, the dead volume has been filled by plain carrier, at which point the plain carrier valve is de-energized. The cycle repeats with a new taggant combination for the second bag and so on with the plain carrier effectively flushing the lines so that only the desired taggants appear for a given lot.

Instead of a spray, the carrier/taggants might be applied by immersion.

The DNA tags can be applied in a complex with a coating composition, for example, a composition including EUDRAGIT® NM 30 D, HPMC (Vivapharm 5), Polysorbate 80 (33% aqueous sol.), talc, and water, which can be coated to a product directly or mixed with another coating material (e.g. carnauba wax ethanol, water, etc., and the like). Studies have shown DNA stability is greater when included in some persistent matrix, as in the presently described complex with EUDRAGIT® NM 30 D, HPMC (Vivapharm 5), Polysorbate 80 (33% aqueous sol.), talc, and water.

As described herein, the final taggant is non-thermally reversible and maintains a homogenous dispersion for a period of time adequate for industrial application of DNA tags. Further, it no longer exhibits the self-binding and agglomeration problems inherent with zein-in-water systems

Encapsulation of the taggant in a carrier, such as a coating solution for a pill or tablet, can provide superior stability. This coating format is convenient for application of taggants to dry and granular products such as tablets, pills, etc.

For commodities such as fertilizers, beans, grains, etc., application of taggants in solid form (encapsulated, e.g. in a matrix such as cyclodextrin) might be preferred due to stability considerations. However, for high speed processes, as when, for example, a taggant must be uniformly applied to product during the bag filling process (which might be a 2-4 second cycle), liquid carriers might be preferred as powder can be very difficult to manage at those speeds and prone to cross contamination. A cost-effective method to apply a taggant in solid form is as very fine powder, which increases the number of taggant particles per volume of product. This increases the probability that the taggant will be recovered from a small sample of product when the product is tested for the presence of the taggants. However, when fine particles are used, they may remain airborne for minutes or even hours, possibly migrating to lots where they were not intended to be applied, which would cause identification errors when taggant reading is done on a sample of the product. Loose particles also might cause cross-lot contamination at the point of testing as the product is taken out of a bag. In these situations, application of the taggants in a liquid form would simplify the application process but might result in diminished stability.

In an improved application process, a hybrid method is used that combines the ease of the liquid application with the stability of the solid carriers. In this approach, the taggant is encapsulated in powder granules, that are suspended in a liquid in which the granules do not dissolve. Examples of encapsulating carriers include gelatins, agarose gel, carrageenan powder, etc. and the liquid carrier might be ethanol. Another example is ethyl cellulose powder as the encapsulating carrier and water as the liquid carrier. The distribution system can then spray a product or immerse the product, thus improving uniform application and reducing the potential for loose powder and resulting cross contamination. The amount of liquid carrier required is usually very small (in one example, less than 50 mL per 50 kg bag). The liquid carrier either evaporates or is absorbed by the product leaving the taggant as an encapsulated powder in the sealed bag.

In addition, use of gels promotes adhesion of the powders to the product, reducing the risk of contamination due to loose powder when the bag is opened. Other adhesives may be added to the liquid to promote adhesion. For example, applying ethyl cellulose powder suspended in a 0.5% agar-agar solution will create a film containing ethyl cellulose DNA tagged powder on the surface of the product.

A dispensing system might include tanks or vessels that contain one of the DNA taggants (or taggants in encapsulating carriers) in suspension, powder, or other forms such as emulsions, liposomes in liquid, or coacervations (a type of electrostatically-driven liquid-liquid phase separation, such as spherical aggregates of colloidal droplets held together by hydrophobic force measuring from 1 to 100 micrometers across or some other diameter, while their soluble precursors are typically on the order of less than 200 nm or some other distance). A computer control system might control the dispensing of specific patterns of the DNA taggants. The taggant vessels have a finite volume and so DNA taggant gets consumed. By careful selection of which patterns are used, the consumption can be controlled so that the taggant vessels do not need to be filled at inconvenient times.

The dispensing system might be required to deliver distinct taggant sets (thus marking distinct objects or lots with different tag strings) at very high speed, as many as 20-25 per minute or more. In an implementation, a computer processor determines what tag string is to be applied and then sends electrical signals and commands to various modules, ultimately resulting in the desired taggant set being added or applied to the object being marked. It may be that each object marked gets a different taggant set, so the dispensing system would carefully control the distribution of taggants so that the taggants of the taggant set applied to a current object do not get used during application of a next object (unless those are taggants that are part of the taggant set for both the current object and the next object).

### Exemplary Implementation #6

In an example implementation, a taggant corresponds to a 28-bit binary word, but in some systems, it could be a 16-bit binary word, a 40-bit binary word, or some other length. Each bit position in the word corresponds to a particular non-coding DNA sequence, such that the presence in the taggant of that particular non-coding DNA sequence is interpreted as the label for that tagged item having a “1” in the bit position of the word that, by perhaps predetermined designation, is assigned to be associated with that particular non-coding DNA sequence.

A non-coding DNA sequence might comprise around 50 to 200 base pairs in a sequence. The taggant might comprise a plurality of the non-coding DNA sequences, in a very low concentration, in a complex with zein, cyclodextrin, casein, and surfactin, which can be coated to a product directly or mixed with another coating material (e.g. carnauba wax ethanol, water, etc., and the like). The particular non-coding sequences (“tags”) might be unique to the environment, such as drawn from seaweed when used for tagging foods or consumer products other than seaweed. The tag sequences might be non-coding, non-viable, non-toxic, generally regarded as safe oligonucleotide. The tag sequences might be microencapsulated in edible particles and/or mixed with carrier liquids. In effect, the collections of tags form “barcodes” by combining multiple DNA tag sequences in unique combinations.

A taggant might be applied during existing production processes, wherein the taggant might comprise the tag sequences in a complex with a coating solution for a pill/tablet, which can be coated to a product (e.g. such as a tablet or pill) directly or mixed with another coating material (e.g. carnauba wax ethanol, water, etc., and the like). In tests, the label (i.e., the binary word encoded by the presence or absence of particular DNA tag sequences in the taggant applied) was readable without error even after 6 months of refrigeration. Where differently labeled products are commingled, correct identification with high reliability is still possible. Stability over time might be as much as several years.

A computer-controlled tank system might be provided with a designated label, and then control which tanks containing tag sequences are opened and tag sequences mixed to form the taggant that is to be applied.

Such a dispensing system might be able to apply a unique label (e.g., a unique DNA barcode) every three seconds, i.e., be able to switch between unique labels in a production process as fast as every three seconds while ensuring that one batch that is supposed to get one label and the next batch that is supposed to get a different label do not get labels “bleeding” over from batch to batch. Where the labels correspond to 28-bit binary words, there are over 250,000,000 possible unique labels. With error correction included, the binary words could be longer or the codeword space could be less than 2\hat{ }28 codewords.

The dispensing system might be used on fruits, nuts, grains, other agricultural products, or other produced materials. With the nature of the taggant, the materials could be bulk granular material, liquids, etc. For example, taggant might be used to label ammonium nitrate fertilizer at the point of production to help track cases of production of improvised ammonium nitrate explosives to determine their source of ammonium nitrate. As the taggant applied is so low volume, it would not be expected to affect the uses of the materials.

The taggant might be applied to equipment and surfaces in consumer safe particles that mimic bacteria behavior. Such particles might attach, detach, transfer and degrade in the presence of sanitizers in the same manner as the target bacteria and the survival of the taggant-laden particles can be tested for at various times. This can easily integrate at scale into existing sanitation and produce wash processes and enable on-site validation, rapid verification, and monitoring of sanitation processes.

A process flow can be described in which the non-coding DNA sequences and taggants might be used. At step 1, data is entered such as lot information and other details pertinent to a batch of material to be labeled with taggant. This might be done via a cloud-connected interface such as a tablet usable on a production floor. At step 2, this information is conveyed to a server that can record the details (for later use in interpreting read labels, for example), authenticate a request and generate an instruction set to be sent to a taggant dispenser that is network-connected. Information might also be recorded in a transaction on a public blockchain so that the instruction set cannot be later altered without detection.

At step 3, the taggant is created from the combination of tag sequences that is consistent with the provided instruction set. At step 4, the taggant is dispensed onto the consumer products or items to be tagged. At step 5, a sample is collected for testing. At step 6, samples can be analyzed using PCR or other techniques. Then, at step 7, the results of the analysis can be provided.

The results of the analysis might be done by, in step 6, first determining which tags were present or absent. Then, as part of step 7, the presence or absence of tags is represented by a binary word and that binary word is used as a lookup (or the information is encoded directly in the binary word) perhaps by reference to the server mentioned in step 2 or by reference to a public blockchain. In that manner, the labeling of a product can be done from source to consumer, regardless of the changes or absence of packaging or conventional labels.

The tags-identity associations can be placed on a public blockchain, thus allowing unrelated parties to check a product in a supply chain, independent of the labels applied by intermediaries and the labeling of consumer products are not limited to labels on the pallet, box, or bag. This would allow third parties to make informed decisions in the event of a recall regarding affected lots, and provides for improved facility and product sanitation based on impact on product quality, shelf life, and safety.

For example, an ingredient producer or a formulator might be running an app on a smartphone or tablet and input into the app details of a product lot (e.g., time of production, name of company, production details, type of ingredient, supervisor name, serial number and network address of their taggant dispenser, etc.). The app might then send those details in a data record to a server that records the details, assigns a unique label (in the form of a binary word, for example, to be used as a DNA “barcode”). The server might also maintain a database of the particular sequences of nucleotides that are in each of the tags that are in the tanks of the taggant dispenser that that grain producer is operating. The server might then send, as a network message, the identified dispenser a listing of the unique label to be used for that batch. Of course, this process might be done for multiple batches at a time, where the ingredient producer or a formulator operator inputs data for several batches and their dispenser receives several unique labels. Since the dispenser is programmed to understand how to mix tags in taggant according to the bits of the unique label, the dispenser can provide taggant that in effect labels the grain with the unique label.

The time of production, name of company, production details, type of grain, supervisor name, batch size, expected customer (if available), serial number of taggant dispenser, the unique label assigned, the code alphabet (which indicates which binary words are valid binary sequences), the error correction used (if any), the taggant suspension and type used (e.g., the coating solution components; additional components can include water, powder, microcapsules, wax, alcohol, etc.) and the sequences of DNA nucleotides used for all of the DNA tags that might have been used in codewords, as well as other details as needed, might all be recorded in one data record that is then inserted into a blockchain transaction and signed by the provider of the taggant dispensers. Once this blockchain transaction is posted to a public blockchain ledger, it cannot be easily altered without others noticing. In this manner, the pertinent details about the labeling using the non-coding DNA sequences are made a public record that any third party could use. For example, suppose a regulator or consumer product safety official traces an illness outbreak to a particular consumer product and there are samples of the consumer product available for testing. The regulator could collect the sample and test it to determine if it was labeled, perhaps by detecting a static portion of non-coding DNA sequences known to be in use. If it was labeled, they could look to the public ledger for a transaction containing the details of the production and without having to resort to research and identifying and getting the cooperation of many different parties in a supply chain can simply look to the blockchain ledger to identify the batch number and producer of the consumer product in question.

In the manner described above, production processes and tracking processes are improved. Examples described herein provide for a method and apparatus for tagging items comprising applying a plurality of non-coding DNA tags, wherein the selection of the particular tags corresponds with a binary or nonbinary code sequence containing information about the tagged items and wherein a non-coding DNA tag comprises a DNA sequence that would not otherwise be present in the tagged item. The items tagged can be consumer products and the information can include information as to a source of the consumer products. The items tagged might be surfaces requiring sanitary handling, where the information includes information as to whether the surfaces were sanitized sufficiently. DNA tags might be selected from among a set of DNA tags with the selection representing and/or corresponding to a label that is a binary word with bits in bit positions corresponding to whether a particular DNA tag was selected. The selection of DNA tags might be combined with a carrier to form a taggant that is applied to surfaces requiring sanitary handling or items to be tagged. The coating solution including the taggant can further include air, water, alcohol or other volatile substance, a wax, a powdering agent, and/or microbeads.

A method for tagging items comprise applying a plurality of non-coding DNA tags, wherein the selection of the particular tags corresponds with a binary or nonbinary code sequence containing information about the tagged items, substantially as shown herein. An apparatus for tagging items might comprise apparatus for applying a plurality of non-coding DNA tags, wherein the selection of the particular tags corresponds with a binary or nonbinary code sequence containing information about the tagged items, substantially as shown herein. An apparatus might be provided for reading tags from tagged items tagged with a plurality of non-coding DNA tags, wherein the selection of the particular tags corresponds with a binary or nonbinary code sequence containing information about the tagged items, substantially as shown herein. Details of the tags might be recorded in a public blockchain transaction.

### Exemplary Implementation #7—Sanitation and Tracing Combined

In an example embodiment, these techniques could be used for tracing a product to its origins or other point in a supply chain it passes through, testing for efficacy of a sanitation process, or both, with information provided in a public manner to allow for independent testing and assessment.

As explained herein and here, a process might start with the selection of a tag to be applied. This tag might be for applying to a product or a surface for later detection without requiring packaging or visible labeling or alteration. The tag might be represented by, and correspond to, a unique sequence of characters. In some embodiments, each character is selected from a binary alphabet, so that the sequence of characters is a bit sequence. In other embodiments, the alphabet has more than two possible characters. An example of such a tag might be a 28-bit, 32-bit, or 60-bit value. In the sequence of characters, each character has a value and a sequence position (e.g., there might be a “1” in the 45th position in the character sequence and a “0” in the seventh position in the character sequence).

Then, there might be a set of noncoding DNA snippets, wherein one of the DNA snippets is associated with one of the character values at one of the character positions. For example, there might be 120 DNA snippets to select from, where 60 DNA snippets are DNA tags for the 60 possible character positions that could have a character value of “1” and 60 other DNA snippets are DNA tags for the 60 possible character positions that could have a character value of “0”. In some variations, some of the character values could be represented by the absence of any of the set of DNA snippets. For example, it could be that there are 28 bit positions in the tag and the set of DNA snippets that make up the DNA tag are 28 DNA snippets and where a character position has a character value of “1”, the corresponding DNA snippet of the 28 DNA snippets is present and when that character position has a character value of “0”, none of the 28 DNA snippets are present. In the general case, where there are M possible characters per character position, and there are N positions, there are MAN possible distinct tags.

A material can be formulated that contains a coating solution, as well as the DNA snippets that correspond to the tag. This material can be applied to a product to be able to trace the product, to a surface to be able to later test for sanitation efficacy, or to a product that is later washed and shipped, to be able to determine both how well it was washed and where it originated.

The tag and additional information about the product or surface can be posted to an unalterable blockchain ledger and at a later time, a sample can be taken from the product or surface, and tested to identify which of the noncoding DNA snippets are present and then the blockchain ledger read to find the blockchain transaction that has the additional information about the product or surface that corresponds to the unique sequence of characters represented by the DNA snippets found in the sample.

### Example 2—Evaluate the Capability of Applying the DNA Barcode Solution

The DNA barcode solution comprise the DNA taggants described herein to pharmaceutical pills using three commercially available coatings and commercially available equipment. The pill weights before and after coating solution application were measured and summarized in Table 2.

TABLE 2
DNA tags concentration applied on each batch of commercial coating solutions.

| | | | | | | | Ave. weight gain per | | |
|---|---|---|---|---|---|---|---|---|---|
| | | | | | | | pill via coating (wet) | | |
| | | | Average | | Ave. weight gain per | | | Average | DNA |
| | | | weight | Std. | pill via coating (Dry) | | Water | Coating | applied |
| Batch | Coating Solution | N | (mg/pill) | Dev. | mg/pill | % | (%) | (mg/pill) | (pg/pill) |
| 0 | Non-coated | 30 | 411.9 | 7.5 | — | — | — | — | — |
| 1 | NM 30 D | 30 | 423.6 | 8.8 | 11.7 | 2.84 | 78 | 53.18 | 148.91 |
| 2 | NM 30 D | 30 | 427.9 | 8.6 | 16.0 | 3.88 | 78 | 72.73 | 203.64 |
| 3 | Eudraguard | 30 | 425.7 | 7.2 | 13.8 | 3.35 | 80 | 68.97 | 193.10 |
| | Biotic | | | | | | | | |
| 4 | Eudraguard | 30 | 423.5 | 6.5 | 11.6 | 2.82 | 70 | 38.67 | 108.27 |
| | Protect | | | | | | | | |

The average dry weight gains are used to re-calculate wet weight gains based on water content in the coating solution. The amount DNA per pill can be calculated using wet weight gain per pill and DNA concentration in coating solution (2.8 pg per mg coating solution), meeting industry requirements.

For each qPCR test strip assay, one pill was transferred using clean forceps to a test tube containing elution solution and suspended by vertex 2-3 seconds. Pill samples were immediately tested in an applied volume of 2 μL to well A B C D E F in a single PCR strip.

The pill test results are shown in Table 3 below. The DNA tags recovery rates from Batches 1 and 2 are excellent, at high double digits between 16.9% and up to 94.5%. The amount of DNA recoverred in batch 3 is between 1.35% and 1.99% which is low when compared to the previous batches. However, the portion of batch 3 that contained the DNA was manufactured 3 days before the pilot, and this specific coating solution is expected to have an acidic pH (2-3) that could have affected the DNA integrity (Toshinori Suzuki, et al., Nucleic Acids Research, 1994, Vol. 22, No. 23 4997-5003). If the values of DNA amount are adjusted using a qPCR measurement of the coating solution used on the day of the pilot, then the amount of DNA on a pill basis was 5.9 pg DNA/pill, resulting in a DNA recovery of 22.9% and 33.7% for sequences D and F respectively, similar to the recovery rates observed in batches 1 and 2.

Batch 4 with coating made of Eudraguard Protect presented lower recovery of DNA, the calculated percent observed is between 0.5% and 2.8% (Table 3), and in terms of mass units the amounts observed are reduced more than 10 times compared the measurements in batch 1 and 2. One possible explanation of this might be that the Eudraguard Protect solution interacted and formed a special structure complex with the DNA tags that could prevent them from releasing from the pill and protects Eudraguard Protect—DNA Tags complex from recapture by swab. Consequently-small amounts of DNA-taggs were recovered from sampled surfaces of the equipment after using this coating.

There is significant carryover of Batch 3 DNA tags into Batch 4 pills, as evidenced by the presence of both Batch 3 and Batch 4 DNA tags in Batch 4 (Table 3). As described earlier, a different cleaning procedure was used before the Batch 4 production that may have contributed to the cross-contamination. The Evonik team attributes this cross-contamination to possible carryover from the mixing blade that was not cleaned between the runs of Batches 3 and 4. The cross-contamination may also be exacerbated by the Eudraguard Protect's ability to interact and form a special structure complex with the contaminated Batch 3 DNA tags. Note that there is no DNA tag cross-contamination and/or carryover observed in Batch 1, 2 and 3 when standard cleaning procedure was used.

TABLE 3
DNA Detection from pills DNA-tagged using different coating formulations.

| | | | | | | Ave. DNA (pg) per | Calculated DNA (pg) | DNA |
|---|---|---|---|---|---|---|---|---|
| | | | | | | pill detected using | per pill using Ave. | recovery |
| Batch | Coating | Sequence | Added in | N | Ave. Ct | qPCR | weight gain per pill | % |
| 1 | NM 30 D | A | + | 100 | 17.3 | 57.23 | 148.91 | 38.4% |
| | | B | + | 100 | 16.7 | 140.67 | 148.91 | 94.5% |
| | | C | − | 100 | 34.8 | 0.00 | — | — |
| | | D | − | 100 | 35.0 | 0.00 | — | — |
| | | E | − | 100 | 35.0 | 0.00 | — | — |
| | | F | − | 100 | 30.7 | 0.01 | — | — |
| 2 | N | A | + | 100 | 18.0 | 34.40 | 203.64 | 16.9% |
| | | B | + | 100 | 17.4 | 84.74 | 203.64 | 41.6% |
| | | C | − | 100 | 34.9 | 0.00 | — | — |
| | | D | − | 100 | 35.0 | 0.00 | — | — |
| | | E | − | 100 | 35.0 | 0.00 | — | — |
| | | F | − | 100 | 31.7 | 0.01 | — | — |
| 3 | Eudraguard | A | − | 101 | 34.9 | 0.00 | — | — |
| | Biotic | B | − | 101 | 34.7 | 0.00 | — | — |
| | | C | − | 101 | 35.0 | 0.00 | — | — |
| | | D | + | 101 | 23.1 | 1.35 | 193.1 | 0.7% |
| | | E | − | 101 | 35.0 | 0.00 | — | — |
| | | F | + | 101 | 24.1 | 1.99 | 193.1 | 1.0% |
| 4 | Eudraguard | A | − | 72 | 32.5 | 0.00 | — | — |
| | Protect | B | − | 72 | 32.2 | 0.01 | — | — |
| | | C | + | 72 | 21.5 | 3.08 | 108.27 | 2.8% |
| | | D | − | 72 | 26.2 | 0.14 | — | — |
| | | E | + | 72 | 21.4 | 0.52 | 108.27 | 0.5% |
| | | F | − | 72 | 28.0 | 0.12 | — | — |

During the testing procedures 10 negative control samples of pills without coating were evaluated for the presence of all the sequences, the results shown 0% false positive detections, with all values of DNA lower than the limit of detection. From the DNA-tagged batches (1-4) a total of 373 DNA-tagged pills were tested for the presence or absence of all DNA tags used. The results are shown in Table 3.

### Example 3 Evaluate the Reliability of Detecting the DNA Barcode Solution on Pharmaceutical Pills Using the qPCR Method

The Ct values obtained (Table 3) were converted to a barcode by a software solution. The barcodes obtained from experimental samples were compared to the expected correct barcode applied in each one of the batches. The results are summarized in the following table.

TABLE 4
DNA-barcode obtained from tested pills.

| | | | | Correct Barcode Detection | | |
|---|---|---|---|---|---|---|
| Batch | Coating | Barcode | Pills Tested N | N | % | 95% C.I. |
| 0 | No Coating | bc-000000 | 10 | 10 | 100% | 69-100% |
| 1 | NM 30 D | bc-110000 | 100 | 100 | 100% | 96-100% |
| 2 | NM 30 D | bc-110000 | 100 | 100 | 100% | 96-100% |
| 3 | Eudraguard Biotic | bc-000101 | 101 | 101 | 100% | 96-100% |
| 4 | Eudraguard Protect | bc-001010 | 72 | 72 | 100% | 95-100% |

The results show that the correct barcode can be detected 100% of the time across all four batches. There are 0 false positive signals from the non-coated negative control pills. Statistically, given the sample size, the proportion of correct barcodes detected is between 95% to 100% for the tagged batches using a 95% confidence interval calculated with a binomial approach (William J. Conover (1971), Practical nonparametric statistics. New York: John Wiley & Sons. Pages 97-104).

### DNA Taggant Detection

Pre-detection amplification might be done by amplifying the sample DNA of the taggants using specific primers with NGS adaptor at 5-end for the known selection set of possible taggants, cycling for 5-20. In some embodiments, pre-detection amplification might be done, by amplifying the sample DNA of the taggants using specific primers with adaptor at 5-end for the known selection set of possible taggants, cycling for 2-5 cycles initially and then continuing to amplify the DNA, using universal adaptor primers labeled with fluorescence dye (FAM) for an additional 30-35 cycles at higher anneal temperature (5-10 C) than initial cycles by continuous flow PCR using static heat blocks at three distinct temperatures. Macrofluidics can be used to cycle the sample over these heat blocks, for rapid ramping. The amplified samples can be subject to next generation sequencing (NGS) using NGS adaptor primers. If the DNA snippet of a taggant is present in the samples, specific taggant sequences are read by NGS. The label's information could be determined from “yes/no” readings of the specific taggant sequences by NGS, possibly in a single test result.

In some embodiments, detection of taggants can be done using an integrated system such as macrofluidic or microfluidic devices with microarray. For example, a microarray might be generated by spotting unique DNA probes complementary to each of DNA snippets of the known selection set of possible taggants (e.g., the 32 possible taggants in the case where N=32 and the label corresponds to a 32-bit value) to each unique position on a microarray. The amplified samples are flowed over a microarray. If the DNA snippet of a taggant is present in the samples, hybridization occurs and fluorescence is detected. The label's information could be determined from “yes/no” readings of the microarray, possibly in a single test result.

In alternative and additional embodiments, detection of taggants can be done in 20 min using a real time PCR instrument containing at least 2×8 well heat block.

In further embodiments, detecting the presence of all 32 bits DNA barcodes on a pharmaceutical product might be done using duplex qPCR in a single test. For example, 16 duplex qPCRs mix, each specific to a pair of 32 bits DNA barcodes, are dispensed to 2×8 well PCR strips. Sample containing DNA snippets of the known selection set of possible taggants (e.g., the 32 possible taggants in the case where N=32 and the label corresponds to a 32-bit value) is applied to all 2×8 wells of PCR strips. The real-time qPCR is performed with these 2×8 well PCR strips, and fluorescence is determined for all 32 possible taggants. The label's information could be determined from “yes/no” readings of the realtime PCR, possibly in a single test result.

In alternative and additional embodiments, detecting the presence of DNA barcodes on a pharmaceutical product might be done using isothermal amplification, such as LAMP or RPA. 32 LAMP mix, each specific to one of 32 bits DNA barcodes, are dispensed to 4×8 well LAMP strips. Sample containing DNA snippets of the known selection set of possible taggants (e.g., the 32 possible taggants in the case where N=32 and the label corresponds to a 32-bit value) is applied to all 32 wells of LAMP strips. The LAMP is performed with these 4×8 well LAMP strips at one elevated constant temperature, and fluorescence is determined for all 32 possible taggants. The label's information could be determined from “yes/no” readings of the LAMP, possibly in a single test result.

In alternative and additional embodiments, detecting the presence of DNA barcodes on a pharmaceutical product might be done using a next-generation DNA sequencer.

### Hardware Implementations

According to one embodiment, the techniques described herein are implemented by one or more generalized computing systems programmed to perform the techniques pursuant to program instructions in firmware, memory, other storage, or a combination. Special-purpose computing devices may be used, such as desktop computer systems, portable computer systems, handheld devices, networking devices or any other device that incorporates hard-wired and/or program logic to implement the techniques.

The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

Further embodiments can be envisioned to one of ordinary skill in the art after reading this disclosure. In other embodiments, combinations or sub-combinations of the above-disclosed embodiments can be advantageously made. The example arrangements of components are shown for purposes of illustration and it should be understood that combinations, additions, re-arrangements, and the like are contemplated in alternative embodiments. Thus, while the invention has been described with respect to exemplary embodiments, one skilled in the art will recognize that numerous modifications are possible.

For example, the processes described herein may be implemented using hardware components, software components, and/or any combination thereof. The specification and drawings are, accordingly, to be regarded in an illustrative rather than a restrictive sense. It will, however, be evident that various modifications and changes may be made thereunto without departing from the broader spirit and scope of the invention as set forth in the claims and that the invention is intended to cover all modifications and equivalents within the scope of the following claims.

All references, including publications, patent applications, and patents, cited herein are hereby incorporated by reference to the same extent as if each reference were individually and specifically indicated to be incorporated by reference and were set forth in its entirety herein.

### Embodiments

Embodiment 1. A method for tagging items comprising:

mixing a plurality of non-coding DNA tags with a solution comprising one or more components, to provide a DNA-containing solution;

introducing the DNA-containing solution to an item to be a tagged item, wherein a selection of particular tags on the tagged item corresponds with a binary or nonbinary code sequence containing information about the tagged item and wherein a non-coding DNA tag comprises a DNA sequence that would not otherwise be present in or on the tagged item; and recording details of the tagged item and details of the particular tags onto a blockchain transaction.

Embodiment 2. The method of embodiment 1, wherein the non-coding DNA tags are encapsulated in the DNA-containing solution.

Embodiment 3. The method of any of embodiments 1-2, wherein mixing the plurality of non-coding DNA tags with a solution comprising one or more components provides a liposomal or micellar structure containing non-coding DNA tags.

Embodiment 4. The method of any of embodiments 1-3, wherein the one or more components comprise pill or tablet coating components, and wherein the tagged item comprises a pill or tablet.

Embodiment 5. The method of embodiment 4, wherein the pill or tablet coating components are coating configured to be used in a pharmaceutical product, a nutraceutical product, a vitamin, or a health or nutritional supplement.

Embodiment 6. The method of any of embodiments 1-6, wherein the non-coding DNA tags comprise a concentration of 1% or less of the DNA-containing solution.

Embodiment 7. The method of any of embodiments 1-6, wherein the tagged item comprises a pharmaceutical product.

Embodiment 8. The method of embodiment 7, wherein the information includes information as to a source of an ingredient of the pharmaceutical product.

Embodiment 9. The method of any of embodiments 1-6, wherein the tagged item comprises a vitamin, health supplement, or nutraceutical product.

Embodiment 10. The method of embodiment 9, wherein the tagged item comprises a vitamin, health supplement, or nutraceutical product, and wherein the information includes information as to a source of an ingredient of the vitamin, health supplement, or nutraceutical product.

Embodiment 11. The method of any of embodiments 1-3, wherein the DNA-containing solution is introduced directly to a formulation or matrix of the item to be a tagged item.

Embodiment 12. The method of embodiments 11, wherein the tagged item comprises a pharmaceutical product, a baby formula, a cosmetic product, a vitamin or supplement, a nutraceutical product, or a personal care product.

Embodiment 13. The method of any of embodiments 1-12, wherein the items are tracked in a supply and/or distribution chain.

Embodiment 14. The method of any of embodiments 1-13, wherein DNA tags are selected from among a set of DNA tags and the selection represents and/or corresponds to a label that is a binary word with bits in bit positions corresponding to whether a particular DNA tag was selected, and wherein the DNA tags of the selection are combined with a carrier to form a taggant that is applied to surfaces requiring sanitary handling or items to be tagged.

Embodiment 15. The method of any of embodiments 1-14, further comprising recording, on a public blockchain, the blockchain transaction and including information related to a tagging and/or labeling process in the blockchain transaction.

Embodiment 16. The method of embodiment 15, wherein the information includes one or more of a time of production, a name of a company, production details, a type of ingredient, a supervisor name, a batch size, an expected customer, a serial number of a taggant dispenser, a label assigned to a batch, a code alphabet, error correction used, a taggant suspension type, and/or sequences of DNA nucleotides used for the plurality of non-coding DNA tags.

Embodiment 17. The method of any of embodiment 1-16, wherein the non-coding DNA tags are in a liposomal or micellar structure in the DNA-containing solution.

Embodiment 18. The method of any of embodiments 1-17, wherein the non-coding DNA tags comprise a taggant material including at least N unique pieces of DNA, representing N digits of a bar code that identifies the taggant material, N being a positive integer greater than 1, wherein each of the at least N unique pieces of DNA represents one value of a corresponding one of the N digits, the method further comprising:

detecting detected pieces of DNA applied to the taggant material;

deriving a derived bar code from the detected pieces of DNA; and

comparing the derived bar code to a predetermined bar code that identifies the taggant material.

Embodiment 19. The method of any of embodiments 1-18, wherein the non-coding DNA tags include at least N unique specific target fragments of synthetic DNA, wherein each of the at least N unique specific target fragments of synthetic DNA corresponds to a binary value of zero or a binary value of one.

Embodiment 20. A DNA-tagged item, comprising:

an item to be a tagged item, in combination with a liposomal or micellar structure containing non-coding DNA tags;

wherein the liposomal or micellar structure containing non-coding DNA tags comprises a plurality of non-coding DNA tags in combination with one or more components in a product formulation or coating solution; and

wherein a selection of particular tags on the tagged item corresponds with a binary or nonbinary code sequence containing information about the tagged item and wherein a non-coding DNA tag comprises a DNA sequence that would not otherwise be present in or on the tagged item.

Embodiment 21. The DNA-tagged item of embodiment 20, wherein the non-coding DNA tags are encapsulated in liposomal or micellar structure.

Embodiment 22. The DNA-tagged item of any of embodiments 20-21, wherein the non-coding DNA tags comprise a concentration of 1% or less of the liposomal or micellar structure.

Embodiment 23. The DNA-tagged item of any of embodiments 20-22, wherein the one or more components in a product formulation or coating solution comprise pill or tablet coating components, and wherein the tagged item comprises a pharmaceutical product, a nutraceutical product, a vitamin, or a health or nutritional supplement, and wherein the tagged item is a tablet or a pill.

Embodiment 24. The DNA-tagged item of any of embodiments 20-23, wherein the tagged item comprises a pharmaceutical product, a nutraceutical product, a vitamin, or a health or nutritional supplement, and wherein the information includes information as to a source of an ingredient of the pharmaceutical product, a nutraceutical product, a vitamin, or a health or nutritional supplement.

Embodiment 25. The DNA-tagged item of any of embodiments 20-24, wherein the liposomal or micellar structure is incorporated directly into a formulation or matrix of the item to be a tagged item.

Embodiment 26. The DNA-tagged item of any of embodiments 20-25, wherein the tagged item comprises a pharmaceutical product, a baby formula, a cosmetic product, a vitamin or supplement, a nutraceutical product, or a personal care product.

Embodiment 27. The DNA-tagged item of any of embodiments 20-26, wherein the items are tracked in a supply and/or distribution chain.

Embodiment 28. The DNA-tagged item of any of embodiments 20-27, wherein DNA tags are selected from among a set of DNA tags and the selection represents and/or corresponds to a label that is a binary word with bits in bit positions corresponding to whether a particular DNA tag was selected, and wherein the DNA tags of the selection are combined with a carrier to form a taggant that is applied to surfaces requiring sanitary handling or items to be tagged.

Embodiment 29. The DNA-tagged item of any of embodiments 20-28, wherein the selection of particular tags on the tagged item is recorded on a blockchain transaction, and wherein the blockchain transaction is recorded on a public blockchain and includes information related to a tagging and/or labeling process in the blockchain transaction.

Embodiment 30. The DNA-tagged item of embodiment 29, wherein the information includes one or more of a time of production, a name of a company, production details, a type of ingredient, a supervisor name, a batch size, an expected customer, a serial number of a taggant dispenser, a label assigned to a batch, a code alphabet, error correction used, a taggant suspension type, and/or sequences of DNA nucleotides used for the plurality of non-coding DNA tags.

Embodiment 31. The DNA-tagged item of any of embodiments 20-30, wherein the non-coding DNA tags comprise a taggant material including at least N unique pieces of DNA, representing N digits of a bar code that identifies the taggant material, N being a positive integer greater than 1, wherein each of the at least N unique pieces of DNA represents one value of a corresponding one of the N digits.

Embodiment 32. The DNA-tagged item of any of embodiments 20-31, wherein the non-coding DNA tags include at least N unique specific target fragments of synthetic DNA, wherein each of the at least N unique specific target fragments of synthetic DNA corresponds to a binary value of zero or a binary value of one.

Embodiment 33. An apparatus for tagging items comprising applying a coating comprising non-coding DNA tags, and coating components, wherein a selection of particular tags corresponds with a binary or nonbinary code sequence containing information about the items.

Embodiment 34. A method of tracking production of a DNA-tagged item, comprising:

obtaining a unique sequence of N characters, N being an integer greater than one, each character having a character value selected from an alphabet of M possible character values, M being an integer greater than one, and each character having a character position within the unique sequence of N characters;

selecting a subset of DNA snippets, to form a DNA tag, from among a set of K*N noncoding DNA snippets, wherein each specific DNA snippet in the subset of DNA snippets is associated with a specific character value and character position and representing the unique sequence of N characters;

combining the DNA tag with one or more coating components to form a DNA-containing coating solution;

applying the DNA-containing coating solution to an item to produce a DNA-tagged item; and

posting a blockchain transaction to a blockchain ledger, wherein the blockchain transaction includes a reference to the unique sequence of N characters and additional information about the item.

Embodiment 35. The method of embodiments 34, further comprising:

obtaining a sample from the DNA-tagged item;

testing the sample to identify which of the K*N noncoding DNA snippets are present on the item; and

read the blockchain ledger to find the blockchain transaction that has the additional information about the item that corresponds to the unique sequence of N characters represented by the DNA snippets found in the sample.

Embodiment 36. The method of any of embodiments 34-35, wherein the DNA-tagged item is a product and the additional information includes an indication of origin of the product, an indication of a path in a supply chain, or a combination thereof.

Embodiment 37. The method of any of embodiments 34-36, wherein the item is a product that is later washed and shipped, and the additional information includes an indication of a sanitation process and details of how the DNA tag was applied to the product prior to the sanitation process and origin information.

Embodiment 38. The method of any of embodiments 34-37, wherein the blockchain transaction includes references to the unique sequence of N characters represented by the DNA snippets of the DNA tag.

Embodiment 39. The method of any of embodiments 34-38, wherein the alphabet is a binary alphabet with each character having one of two possible character values.

Embodiment 40. The method of any of embodiments 34-39, wherein K is equal to M−1 and one character value in a given character position is represented in the DNA tag by absence of a noncoding DNA snippets of the set of K*N noncoding DNA snippets that is assigned to that given character position.

Embodiment 41. The method of embodiment 40, wherein the character values are “0” and “1” and the one character value is “0”, whereby that one character value of “0” in the given character position is represented in the DNA tag by absence of a specific noncoding DNA snippet is assigned to that given character position.

Embodiment 42. The method of embodiment 41, wherein the subset of DNA snippets comprises, for each character position, one of M possible DNA snippets or one of M−1 possible DNA snippets with the absence of a DNA snippet for that character position encoding for one character value, thereby encoding the unique character sequence into one of MAN distinct DNA tags.

What is claimed is:

1. A method for tagging items comprising:
mixing a plurality of non-coding DNA tags with a solution comprising one or more components, to provide a DNA-containing solution;
introducing the DNA-containing solution to an item to be a tagged item, wherein a selection of particular tags in or on the tagged item corresponds with a binary or nonbinary code sequence containing information about the tagged item and wherein a non-coding DNA tag comprises a DNA sequence that would not otherwise be present in or on the tagged item; and
recording details of the tagged item and details of the particular tags onto a blockchain transaction.

2. The method of claim 1, wherein the non-coding DNA tags are encapsulated in the DNA-containing solution.

3. The method of claim 1, wherein mixing the plurality of non-coding DNA tags with a solution comprising one or more components provides a liposomal or micellar structure containing non-coding DNA tags.

4. The method of claim 1, wherein the one or more components comprise pill or tablet coating components, and wherein the tagged item comprises a pill or tablet.

5. The method of claim 4, wherein the pill or tablet coating components are coating configured to be used in a pharmaceutical product, a nutraceutical product, a vitamin, or a health or nutritional supplement.

6. The method of claim 1, wherein the non-coding DNA tags comprise a concentration of 1% or less of the DNA-containing solution.

7. The method of claim 1, wherein the tagged item comprises a pharmaceutical product.

8. The method of claim 7, wherein the information about the tagged item includes information as to a source of an ingredient of the pharmaceutical product.

9. The method of claim 1, wherein the items are tracked in a supply and/or distribution chain.

10. The method of claim 1, wherein DNA tags are selected from among a set of DNA tags and the selection represents and/or corresponds to a label that is a binary word with bits in bit positions corresponding to whether a particular DNA tag was selected, and wherein the DNA tags of the selection are combined with a carrier to form a taggant that is applied to surfaces requiring sanitary handling or items to be tagged.

11. The method of claim 1, further comprising recording, on a public blockchain, the blockchain transaction and including information related to a tagging and/or labeling process in the blockchain transaction.

12. The method of claim 1, wherein the non-coding DNA tags include at least N unique specific target fragments of synthetic DNA, wherein each of the at least N unique specific target fragments of synthetic DNA corresponds to a binary value of zero or a binary value of one.

13. A DNA-tagged item, comprising:
an item to be a tagged item, in combination with a liposomal or micellar structure containing non-coding DNA tags;
wherein the liposomal or micellar structure containing non-coding DNA tags comprises a plurality of non-coding DNA tags in combination with one or more components in a product formulation or coating solution; and
wherein a selection of particular tags on the tagged item corresponds with a binary or nonbinary code sequence containing information about the tagged item and wherein a non-coding DNA tag comprises a DNA sequence that would not otherwise be present in or on the tagged item.

14. The DNA-tagged item of claim 13, wherein DNA tags are selected from among a set of DNA tags and the selection represents and/or corresponds to a label that is a binary word with bits in bit positions corresponding to whether a particular DNA tag was selected, and wherein the DNA tags of the selection are combined with a carrier to form a taggant that is applied to surfaces requiring sanitary handling or items to be tagged.

15. The DNA-tagged item of claim 13, wherein the selection of particular tags on the tagged item is recorded on a blockchain transaction, and wherein the blockchain transaction is recorded on a public blockchain and includes information related to a tagging and/or labeling process in the blockchain transaction.

16. The DNA-tagged item of claim 15, wherein the information related to a tagging and/or labeling process includes one or more of a time of production, a name of a company, production details, a type of ingredient, a supervisor name, a batch size, an expected customer, a serial number of a taggant dispenser, a label assigned to a batch, a code alphabet, error correction used, a taggant suspension type, and/or sequences of DNA nucleotides used for the plurality of non-coding DNA tags.

17. The DNA-tagged item of claim 13, wherein the non-coding DNA tags comprise a taggant material including at least N unique pieces of DNA, representing N digits of a bar code that identifies the taggant material, N being a positive integer greater than 1, wherein each of the at least N unique pieces of DNA represents one value of a corresponding one of the N digits.

18. The DNA-tagged item of claim 13, wherein the non-coding DNA tags include at least N unique specific target fragments of synthetic DNA, wherein each of the at least N unique specific target fragments of synthetic DNA corresponds to a binary value of zero or a binary value of one.

19. A method of tracking production of a DNA-tagged item, comprising:
obtaining a unique sequence of N characters, N being an integer greater than one, each character having a character value selected from an alphabet of M possible character values, M being an integer greater than one, and each character having a character position within the unique sequence of N characters;
selecting a subset of DNA snippets, to form a DNA tag, from among a set of K*N noncoding DNA snippets, wherein each specific DNA snippet in the subset of DNA snippets is associated with a specific character value and character position and representing the unique sequence of N characters;
combining the DNA tag with one or more coating components to form a DNA-containing coating solution;
applying the DNA-containing coating solution to an item; and
posting a blockchain transaction to a blockchain ledger, wherein the blockchain transaction includes a reference to the unique sequence of N characters and additional information about the item.

20. The method of claim 19, further comprising:
obtaining a sample from the DNA-tagged item;
testing the sample to identify which of the K*N noncoding DNA snippets are present on the item; and
read the blockchain ledger to find the blockchain transaction that has the additional information about the item that corresponds to the unique sequence of N characters represented by the DNA snippets found in the sample.
